(19) United States
(12) Patent Application Publication
(10) Pub. No.: US 2017/0290849 A1
(43) Pub. Date: Oct. 12, 2017

(54) AKT and SHH Pathway Inhibitor Combination Therapy for Treating Basal Cell Carcinomas

(71) Applicant: THE TRUSTEES OF COLUMBIA UNIVERSITY IN THE CITY OF NEW YORK, New York, NY (US)

(72) Inventors: Arianna L. Kim, Palisades Park, NJ (US); David Bickers, Larchmont, NY (US); Mohammad Athar, Hoover, AL (US)

(73) Assignee: The Trustees of Columbia University in the City of New York, New York, NY (US)

(21) Appl. No.: 15/508,571

(22) PCT Filed: Sep. 8, 2015
(86) PCT No.: PCT/US15/49044

§ 371 Date: Mar. 3, 2017

Related U.S. Application Data
(60) Provisional application No. 62/046,889, filed on Sep. 5, 2014.

(51) Int. Cl. A61K 31/685; A61K 9/00; A61K 31/496; A61K 31/436; A61K 31/4375; A61K 31/4355; A61K 31/4418; A61K 31/5377; A61K 31/502; A61K 31/454; A61K 31/506; A61K 31/517; A61K 45/06; A61K 31/519

(52) U.S. Cl. CPC A61K 31/685; A61K 45/06; A61K 9/0019; A61K 9/0053; A61K 31/496; A61K 31/436; A61K 31/4375; A61K 31/519; A61K 31/4418; A61K 31/5377; A61K 31/502; A61K 31/454; A61K 31/506; A61K 31/517; A61K 31/4355

(57) ABSTRACT

A mechanism whereby aberrant Shh signaling converges on the Akt1-mTOR pathway, conferring selective growth advantage and enhanced survival of tumor cells has been identified. Utilizing a mouse model of BCNS, a pivotal role has been discovered for Akt1 signaling in BCC tumorigenesis. Based on the results described here certain embodiments are directed to methods and pharmaceutical formulations for treating BCC/BCNS, other cancers that are Shh+ and Akt+, cancers that are Shh+ and mTOR plus and cancers that are Shh+ by administering therapeutically effective amounts of various combinations of Akt inhibitors, Shh pathway inhibitors such as SMO inhibitors, and mTOR inhibitors.

### CROSS REFERENCE TO RELATED APPLICATIONS

This application claims benefit of U.S. Provisional Application No. 62/046,889, filed Sep. 5, 2014, the entire contents of each of which are hereby incorporated by reference as if fully set forth herein, under 35 U.S.C. §119(e).

### STATEMENT OF GOVERNMENT INTEREST

This invention was made with government support under Grant No. R01ES020344 awarded by the National Institutes of Health. The government has certain rights in the invention.

### BACKGROUND

Basal cell carcinomas (BCCs) of the skin are the most common type of human malignancy. Similar to other skin cancers, the risk of BCCs is inversely correlated to skin pigmentation and directly correlated to exposure to UV radiation in sunlight. These tumors, therefore, occur predominantly on body areas that are sun-exposed. Patients affected with the rare dominantly inherited disorder known as basal cell nevus syndrome (BCNS) or Gorlin's syndrome develop tens to hundreds of BCCs in greater abundance on sun-exposed sites, as well as various extra cutaneous tumors. The sonic hedgehog (Shh) signaling pathway genes, such as Patched (Ptch) and smoothened (SMO) are critical components in embryonic development, and Ptch mutations are linked to BCC induction.

Germline Ptch 1 mutations in the sonic hedgehog (Shh) signaling pathway drive the spontaneous growth of basal cell carcinomas (BCC) in Gorlin/nevoid basal cell carcinoma syndrome (BCCNS). However, Shh-targeted therapies in these patients—have proven to be less than ideal due to tumor recurrence, coupled with the acquisition of drug-resistant secondary mutations in smoothened (SMO)—suggesting that additional mechanisms underlie BCC carcinogenesis.

### SUMMARY

Certain embodiments of the invention are directed to methods for treating basal cell carcinoma, basal cell nevus syndrome cancer, or XP BCC, comprising administering a therapeutically effective amount of an Akt inhibitor. Another embodiment further comprises administering a therapeutically effective amount of an mTOR inhibitor or a sonic hedgehog inhibitor together with Akt inhibitor; and yet another embodiment further comprises administering a sonic hedgehog pathway inhibitor, and mTOR inhibitor together with the Akt inhibitor. Another embodiment is directed to a method for treating a cancer that has activated sonic hedgehog and Akt (Shh+/Akt+), comprising administering a therapeutically effective amount of a sonic hedgehog pathway inhibitor and an Akt inhibitor, or therapeutically effective amount of a sonic hedgehog pathway inhibitor and an mTOR inhibitor, a combination thereof all three inhibitors. Another embodiment is directed to a method for treating a cancer that has activated sonic hedgehog and mTOR(Shh+/mTOR+), comprising administering a therapeutically effective amount of a sonic hedgehog pathway inhibitor and an mTOR inhibitor, or a combination of a sonic hedgehog inhibitor, an Akt inhibitor and an mTOR inhibitor.

In some embodiments the Akt inhibitor is selected from the group consisting of perifosine, edelfosine, MK-2206 and AZD5363 or pharmaceutically active derivatives thereof. In other embodiments the sonic hedgehog pathway inhibitor is a smoothened inhibitor including a member selected from the group consisting of vismodegib and itraconazole, an agent listed in Table 1 or Table 2, or pharmaceutically active derivatives thereof. In some embodiments the mTOR inhibitor is selected from the group consisting of rapamycin Agent OSI-027, XL765, everolimius, temsirolimus and zotarolimus, or pharmaceutically active derivatives thereof.

Sonic hedgehog- and Akt− activated cancers (Shh+/Akt+), and sonic hedgehog- and mTOR-activated cancers (Shh+/mTOR) include colon, pancreas, medulloblastoma, prostate, esophageal, glioma, and gastrointestinal cancers.

Certain embodiments are directed to pharmaceutical compositions, comprising a therapeutically effective amount of an Akt1 inhibitor together with a sonic hedgehog pathway inhibitor; or comprising a therapeutically effective amount of an Akt1 inhibitor together with a mTOR inhibitor, or comprising an Akt1 inhibitor together with a sonic hedgehog pathway inhibitor and a mTOR inhibitor. In an embodiment the pharmaceutical composition is formulated for topical or oral administration or by injection.

In other embodiments the therapeutic methods administer a dose of each named active agent in an amount of from about 0.1 mg/day to about 1 gm/day, an amount of about 1-25 mg/day, 25-50 mg/day, 50-100 mg/day, 100-200 mg/day, 200-300 mg/day, 400-500 mg/day and 500-1000 mg/day.

In other embodiments the pharmaceutical formulations comprise an amount of each named active agent of from about 1-25 mg, 25-50 mg, 50-100 mg, 100-200 mg, 200-300 mg, 400-500 mg and 500-1000 mg.

These and other features, aspects, and advantages of the present invention will become better understood with regard to the following description, appended claims, and accompanying figures.

### BRIEF DESCRIPTION OF THE DRAWINGS

The following drawings form part of the present specification and are included to further demonstrate certain embodiments of the present invention. The invention may be better understood by reference to one or more of these drawings in combination with the detailed description of specific embodiments presented herein.

FIGS. 1A-1E are photographs and graphs showing that Akt1 is activated in BCCs and fosters the survival of human keratinocytes. FIG. 1A: Total and phospho-Akt1 (S473) expression was assessed in spontaneous and UV-induced BCCs in Ptchl^{+/-}/SKH-1 mice and sporadic human BCCs by Western blotting. FIG. 1B: Immunohistochemical assessment of Akt1 and p-Akt1 (S473) in non-irradiated skin (FIG. 1Ba, FIG. 1Be), spontaneously developed (FIG. 1Bb, FIG. 1Bf) and UV-induced (FIG. 1Bc, FIG. 1Bg) BCCs in Ptch1^{+/-}/SKH-1 mice, and in human BCCs (FIG. 1Bd, FIG. 1Bh). FIG. 1C: Expression of constitutively active Akt1 (pUSEamp-myrAKT1, myrAkt1) and Gli2 (Gli2ΔN) leads to increased incorporation of BrdU in normal human keratinocytes. FIG. 1D: Overexpression of myrAkt1 protects human keratinocytes from UV-induced apoptosis. FIG. 1E: Colony forming capability of pBAP (−) or myr-Akt1-expressing normal human keratinocytes. *P<0.005, compared to control.

FIGS. 2A-2F are graphs showing inhibition of the Shh pathway by itraconazole suppresses Akt1 signaling in vitro and in vivo. FIG. 2A: Itraconazole decreases the viability of murine BCC (ASZ001) cells. *p<0.05. FIG. 2B: Pathway components of Shh and Akt1 signaling decrease in ASZ001 cells treated with itraconazole. FIG. 2C, FIG. 2D: mRNA levels of mTOR and cyclin D1 were measured by qPCR in itraconazole-treated ASZ001 cells. Shh and Akt-mTOR pathway components were reduced (FIG. 2E) and number of microscopic BCCs decreased (FIG. 2F) in itraconazole-treated mice.

FIGS. 3A-3D are graphs and photographs showing SOX9 is overexpressed in BCCs and its knockdown decreases mTOR signaling. FIG. 3A: SOX9 is overexpressed in both human and UV-induced murine BCCs. SOX9 expression was assessed in UV-induced murine BCCs and human sporadic BCCs by Western blotting. FIG. 3B: Densitometric scanning of FIG. 3A. FIG. 3C: Immunohistochemical distribution of SOX9 in BCCs. 100× magnification. FIG. 3D: shRNA-mediated knockdown of SOX9 downregulates mTOR signaling as assessed by Western blotting of ASZ001 cells stably transfected with pRK-shGFP or pRK-shSOX9.

FIGS. 4A-4E are graphs showing SOX9 transcriptionally regulates mTOR. FIG. 4A: Consensus SOX9-binding motifs are found in the mouse mTOR promoter. Nuclear extracts prepared from murine BCC (ASZ001) cells bind to three putative SOX9-binding motifs and SOX9 knockdown (shSOX9) reduces this binding at −4067 (FIG. 4B), as assessed by gel-shift assays. FIG. 4C: Recombinant mouse SOX9 proteins bind to the putative SOX9-binding motifs. FIG. 4D: Occupancy of consensus SOX9 sites at −4069 and −9313 bp within the mTOR promoter analyzed by ChIP analysis. FIG. 4E: mTOR promoter transactivation in ASZ001 cells. *p<0.05.

FIGS. 5A-5D are graphs and photographs showing Akt1 deficiency blocks the development of spontaneous microscopic BCCs in Akt1^{+/-}/Ptch1^{+/-}/SKH-1 mice. FIG. 5A: SOX9, mTOR, and p-Akt1 (S473) levels are increased in primary keratinocytes isolated from postnatal day 2 Ptch1^{+/-} mice (lanes 3 and 4), but not in primary keratinocytes isolated from postnatal day 2 Ptch1^{+/-} mice (lanes 1 and 2). FIG. 5B: Representative H&E staining of skin sections from Akt1^{+/-}/Ptch1^{+/-}/SKH-1 mice and Ptch1^{+/-}/SKH-1 littermates. Scale bar=200 μm. FIG. 5C: Assessment of size and number of spontaneous BCCs in Akt1^{+/-}/Ptch1^{+/-}/SKH-1 and Ptch1^{+/-}/SKH-1. FIG. 5D: The levels of Akt isoforms in Akt1^{+/-}/Ptch1^{+/-}/SKH-1 mice and Ptch1^{+/-}/SKH-1 littermates, assessed by Western blotting.

FIGS. 6A-6D are photographs and graphs showing Akt1 deficiency suppresses UV-induced growth of BCCs in Akt1^{+/-}/Ptch1^{+/-}/SKH-1 mice. Ptch1^{+/-}/SKH-1 (n=6) and Akt1^{+/-}/Ptch1^{+/-}/SKH-1(n=5), and their wild type littermates (n=5) were irradiated with UV (180 mJ/cm², twice weekly for 30 weeks. FIG. 6A: Representative H&E staining of skin sections harvested at week 30. FIG. 6B: Akt1 deficiency suppresses UV-induced skin tumors. FIG. 6C: Average tumor volume per animal. FIG. 6D: Microscopic BCCs. Each dot represents data from one mouse.

FIGS. 7A-7D are graphs showing inhibition of Akt signaling with the alkylphospholipid drugs perifosine or edelfosine suppresses UV-induced growth of BCCs. FIG. 7A: Alkylphospholipids reduce Akt phosphorylation and induce apoptosis in ASZ001 cells. FIG. 7B: Akt inhibition reduces proliferation of ASZ001 cells. FIG. 7C: Orally administered perifosine prevents the growth of UV-induced BCCs and (FIG. 7D) microscopic BCCs in Ptch1^{+/-}/SKH-1 mice. Each dot represents data from one mouse.

FIGS. 8A-8C graphs showing perifosine combined with SMO inhibitors acts synergistically in vivo. FIG. 8A: In both ASZ001 and myrAkt1 transformed ASZ001 cells, treatment regimens with perifosine reduced phosphorylation of endogenous Akt at T308 and S473. (black arrowheads indicate myrAkt1Δ4-129). FIG. 8B: In both ASZ001 (top) and myrAkt: ASZ001 (bottom), combination regimens were more effective at inhibiting proliferation than single treatments alone. FIG. 8C: A synergistic effect of combination therapy on increasing apoptosis was observed when the Akt inhibitor perifosine was administered with itraconazole.

FIGS. 9A-9H are photographs and graphs showing perifosine combined with SMO inhibitors acts synergistically in vivo. FIG. 9A-FIG. 9F shows Ptch1^{+/-} /SKH-1 hairless mice that were UV-irradiated (180 mJ/cm2, twice weekly for 4 weeks) and treated with perifosine (50 mg/kg, PO BID, oral) combined with either itraconazole or vismodegib (100 mg/kg, PO BID, oral). Each dot in FIG. 9G and FIG. 9H represent data from one skin strip. Three skin strips (avg. 1.5 cm×0.1 cm) were analyzed for each mouse.

### DETAILED DESCRIPTION

The results presented here delineate the mechanism whereby aberrant Shh signaling converges on the Akt1-mTOR pathway, conferring selective growth advantage and enhanced survival of tumor cells. Utilizing a mouse model of BCNS (Chaudhary S C, In press #2833), a pivotal role has been discovered for Akt1 signaling in BCC tumorigenesis. Based on the results described here, certain embodiments are directed to methods and pharmaceutical formulations for treating BCC/BCNS, other cancers that are Shh+ and Akt+, cancers that are Shh+ and mTOR plus and cancers that are Shh+ by administering therapeutically effective amounts of various combinations of Akt inhibitors Shh pathway inhibitors such as SMO inhibitors and mTOR inhibitors.

In the following description, for the purposes of explanation, numerous specific details are set forth in order to provide a thorough understanding of the present invention. It will be apparent, however, to one skilled in the art that the present invention may be practiced without these specific details. In order that the invention may be readily understood and put into practical effect, particular preferred embodiments will now be described by way of the following non-limiting examples.

### 1. Definitions

Unless otherwise defined, all technical and scientific terms used herein are intended to have the same meaning as commonly understood in the art to which this invention pertains and at the time of its filing. Although various methods and materials similar or equivalent to those described herein can be used in the practice or testing of the present invention, suitable methods and materials are described below. However, the skilled should understand that the methods and materials used and described are examples and may not be the only ones suitable for use in the invention. Moreover, it should also be understood that as measurements are subject to inherent +variability, any temperature, weight, volume, time interval, pH, salinity, molarity or molality, range, concentration and any other measurements, quantities or numerical expressions given herein are intended to be approximate and not exact or critical figures unless expressly stated to the contrary. Hence, where appropriate to the invention and as understood by those of skill in the art, it is proper to describe the various aspects of the invention using approximate or relative terms and terms of degree commonly employed in patent applications, such as: so dimensioned, about, approximately, substantially, essentially, consisting essentially of, comprising, and effective amount.

Generally, nomenclature used in connection with, and techniques of, cell and tissue culture, molecular biology, immunology, microbiology, genetics, protein, and nucleic acid chemistry and hybridization described herein are those well-known and commonly used in the art. The methods and techniques of the present invention are generally performed according to conventional methods well known in the art and as described in various general and more specific references that are cited and discussed throughout the present specification unless otherwise indicated. See, e.g., Sambrook et al. Molecular Cloning: A Laboratory Manual, 2d ed., Cold Spring Harbor Laboratory Press, Cold Spring Harbor, N.Y. (1989); Ausubel et al., Current Protocols in Molecular Biology, Greene Publishing Associates (1992, and Supplements to 2002); Harlow and Lan, Antibodies: A Laboratory Manual, Cold Spring Harbor Laboratory Press, Cold Spring Harbor, N. Y. (1990); Principles of Neural Science, 4th ed., Eric R. Kandel, James H. Schwartz, Thomas M. Jessell editors. McGraw-Hill/Appleton & Lange: New York, N. Y. (2000). Unless defined otherwise, all technical and scientific terms used herein have the same meaning as commonly understood by one of ordinary skill in the art.

Abbreviations: BCC, basal cell carcinoma; BCNS=basal cell nevus syndrome/ Gorlin; Dhh, desert hedgehog; GLI1, glioma-associated oncogene homolog 1; Hedgehog, Hh; IGF-2, insulin-like growth factor 2; Ihh, Indian hedgehog; mTOR, mammalian target of rapamycin; PTCH, patched; PI3K/Akt, phosphatidylinositol 3-kinase/v-akt murine thymoma viral oncogene homologue; SMO, smoothened; SOX9, Sry-related HMG box 9; UV, ultraviolet.

The term “Akt” as used herein means Protein kinase B (PKB), which is a serine/threonine-specific protein kinase that plays a key role in multiple cellular processes such as glucose metabolism, apoptosis, cell proliferation, transcription and cell migration. Akt as used herein includes all three isoforms of Akt: Akt-1, -2, and -3. Certain cancers are identified by activation of various isoforms of Akt. Akt1 is activated in BCC that also develop in BCNS patients.

The term “Akt-1” as used herein means a gene that encodes RAC-alpha serine/threonine-protein kinase. This enzyme belongs to the AKT subfamily of serine/threonine kinases that contain SH2 (Src homology 2-like) domains. It is commonly referred to as PKB or by both names as “Akt/PKB.” Akt-1 is an isoform of Akt serine/threonine protein kinase that is directly regulated by the Shh pathway transcription factor SOX9 via mTOR. This Akt also confers resistance to UV-induced apoptosis, and activation of Akt1 is pivotal in BCC tumorigenesis.

The term “Akt inhibitor” as used herein means any inhibitor that blocks or reduces activity of the Akt protein and includes perifosine and edelfosine and others described herein. Because of the homology of the various Akt isoforms as well as the mechanisms of action of Akt inhibitors, an Akt inhibitor typically is effective in inhibiting the activity of all of the isoforms, though perhaps to varying degrees.

The terms “Akt+ tumor/cancer” and “Akt-activated tumor/cancer” as used herein are used interchangeably and mean one in which any isoform of the Akt enzyme has been activated (i.e. phosphorylated).

The terms “active agents” and “therapeutic agents” as used herein are interchangeable and mean any agent (synthetic, recombinant or natural) used in embodiments of the invention, including Akt1 inhibitors, mTOR inhibitors and Shh pathway inhibitors (SPI) like SMO inhibitors described herein and others known to persons of skill in the art.

The terms “administering” or “administration of” as used herein mean a drug or therapeutic pharmaceutical composition to a subject any method known in the art includes both direct administration, including self-administration (including oral administration or intravenous, subcutaneous, intramuscular or intraperitoneal injections, rectal administration by way of suppositories), local administration directly into or onto a target tissue (such as topical administration in the case of BCC) or administration by any route or method that delivers a therapeutically effective amount of the drug or composition to the cancer cells, tumors or tissue to which it is targeted.

The terms “tumor and cancer” as used herein are used interchangeably.

The terms “Akt+ and Shh+ cancers (Akt+/Shh+)” as used herein mean cancers in which both Akt and Shh are activated. Activation of Akt1 and Shh is pivotal in BCC tumorigenesis. Embodiments of the invention provide therapies for treating cancers that are Akt+/Shh+, including BCC as well as colon, pancreas, medulloblastoma, prostate, esophageal, glioma, and gastrointestinal cancers. Morton J P, Lewis B C (2007), “SHH signaling and pancreatic cancer: implications for therapy?” Cell Cycle 6 (13): 1553-7; Ther Adv Med Oncol. 2010 July; 2(4): 237-250.

The term “Akt-mTOR pathway” as used herein means the pathway from mTOR to Akt, or from Akt to mTOR.

The term “ASZ001” as used herein means cultured murine BCC cells. Akt inhibition concomitantly reduced activating Akt phosphorylation and proliferation of cultured BCC (ASZ001) cells to varying degrees, as assessed by BrdU incorporation. Of these, only the alkyl-lysophospholipid analogs perifosine and edelfosine induced apoptosis, as shown by increased cleaved caspase-3. Thus these two agents reduced proliferation and induced apoptosis of BCC.

The terms “basal cell carcinoma or basal cell cancer (BCC)”, as used herein, mean a skin cancer, and is one of the most common cancers in the United States. It rarely metastasizes or kills However, because it can cause significant destruction and disfigurement by invading surrounding tissues, it is still considered malignant. BCC as used herein includes BCCs developed in both general population and individuals affected with BCNS. Multiple BCCs develop in individuals affected with BCNS. BCCs are Akt+ tumors that also have activated mTOR in addition to activated Shh+ pathways. Therefore they can be treated with an mTOR or Akt inhibitor such as perifosine alone, or more effectively and Akt inhibitor plus Shh inhibitors.

The term “Basal Cell Nevus Syndrome (BCNS)” as used herein means Gorlin Syndrome, Gorlin-Goltz Syndrome, or Nevoid Basal Cell Carcinoma Syndrome. BCNS is a rare genetic disorder that may affect all systems of the body and makes affected individuals more susceptible to various forms of cancer, especially basal cell carcinoma, a form of skin cancer. BCC that develops in BCNS patients is a type of Akt+ tumor.

The term “itraconazole” as used herein means a FDA-approved azole antifungal drug that is a potent and specific inhibitor of Shh signaling by inhibiting smoothened (SMO). As used herein itraconazole is interchangeably referred to as an SMO inhibitor or another Shh pathway inhibitor.

The term “mammalian target of rapamycin (mTOR)” as used herein means the serine/threonine protein kinase that regulates cell growth, cell proliferation, cell motility, cell survival, protein synthesis, autophagy, and transcription. [3] MTOR belongs to the phosphatidylinositol 3-kinase-related kinase protein family. For the purpose of the embodiments herein described, mTOR is part of the Akt-mTOR pathway and the Shh pathway. The Shh pathway component SOX9 directly regulates mTOR in BCC. So, mTOR is part of the Shh pathway in BCCs; which is a new discovery and provides a basis for using Akt inhibitors to treat Shh+ tumors, including BCC and others.

The term “mTOR+” cancers as used herein includes those in which mTOR is overexpressed/activated.

The term “mTOR” inhibitors as used herein means drugs that inhibit mTOR activity, which include rapamycin (also known as Sirolimus), Agent OSI-027 (OSI Pharmaceuticals, Melville, N.Y., U.S.A.) that is currently in phase 1 of trial and being evaluated on patients with lymphoma or solid tumors, XL765 (Exelixis, San Francisco, Calif., U.S.A.) that is also in phase 1 of clinical trial and being assessed in combination therapies, and three other molecules already approved for therapeutic use (Everolimius, Temsirolimus (approved for renal cell carcinoma) and Zotarolimus. See Baldo P Curr Cancer Drug Targets. 2008 December; 8(8):647-65; Riaz et al. Infectious Agents and Cancer 2012, 7:1. See also Table 1.

The term “pharmaceutically acceptable” as used herein means a carrier, diluent or excipient must be compatible with the other ingredients of the formulation and not deleterious to the recipient thereof.

The term “smoothened (SMO)” as used herein means an oncogene that in humans is encoded by the SMO gene. The abbreviation “SMO” as used herein refers to either the oncogene or the encoded protein, depending on the context. Smoothened is a G protein-coupled receptor that is a component of the hedgehog signaling pathway and is conserved from flies to humans.

The term “smoothened (SMO) inhibitor” as used herein means includes inhibitors of SMO protein activity, such as vismodegib and itraconazole and others listed in Table 1. Often SMO inhibitors are called “sonic hedgehog inhibitors” in the literature.

The term “sonic hedgehog (Shh) pathway” means sonic hedgehog or hedgehog pathway, and as used herein includes Shh, PTCH, SMO, GLI1-3, SOX9, and downstream targets.

The term “sonic hedgehog” or “Shh” as used herein is a ligand for PTCH protein that in humans is encoded by the sonic hedgehog gene. The abbreviation “Shh” as used herein refers to either the gene or the protein, depending on the context. Sonic hedgehog is one of three proteins in the mammalian signaling pathway family called hedgehog, the others being desert hedgehog (DHH) and Indian hedgehog (IHH).

The terms “sonic hedgehog-positive (Shh+)” and “Shh activated cancer” are used interchangeably to mean any cancer in which the Shh pathway has been activated. The Shh pathway can be activated in different ways. One way is through activation of the smoothened (SMO) oncogene and another way is through the inactivation of the tumor suppressor PATCHED (PTCH). In some Shh+ tumors, Gli is activated by mutation or amplification, which also drives Shh pathway signaling. All of these changes have the potential to cause a tumor. Shh+ cancers include BCC.

The term “Shh pathway activation” as used herein includes, but is not limited to loss-of-function mutations in the tumor suppressor PTCH1, or gain-of-function mutations in the oncogene SMO, and overexpression or missense mutations in GLI1 and GLI3, that can lead to cancer. Germline mutations of the PTCH are found in the BCNS patients. Sporadic BCCs in general population and BCCs in xeroderma pigmentosum (XP) patients have mutations of PTCH as well as SHH and SMO. XP is an autosomal recessive genetic disorder of DNA repair in which the ability to repair damage caused by ultraviolet (UV) light is deficient.

The term “Shh pathway inhibitor (SPI)” as used herein means any naturally derived or synthetic compounds that can inhibit the activating signaling of the Shh pathway and includes SMO inhibitors. They can include agents that inhibit SHH or Gli. It can also include agents that increase the tumor suppressor PTCH1 activity.

The term “sonic hedgehog/mTOR activated cancer” as used herein means a cancer wherein an element in the Shh pathway is activated and mTOR is activated.

The term “sonic hedgehog (Shh) inhibitor” as used herein any inhibitors that inhibit Shh pathway signaling and include agents that inhibit SHH (including the Shh inhibitor Robotnikinin), SMO, or GLI.

The term “SOX9” as used herein is a transcription factor belongs to group E of the SOX transcription factor family (SOX8, SOX9, and SOX10) defined by a common HMG box domain originally identified in SRY, the sex-determining gene on the Y chromosome. SOX9 has been shown to influence epithelial cell proliferation and migration in developing prostate and to have similar roles in prostate cancer. It is also a master factor regulating chondrocyte development. SOX9 is upregulated in BCCs and the Shh pathway downstream transcription factor, GLI-1, regulates SOX9 expression.

The terms “subject” or “patient” as used herein mean a mammal, typically a human, but optionally a mammalian animal of veterinary importance, including but not limited to horses, cattle, sheep, dogs, and cats.

The term “therapeutically effective amount” as used herein means an amount of an active agent or pharmaceutical composition that achieves the intended therapeutic effect, e.g., alleviation, amelioration, palliation or elimination of one or more manifestations of the disease or condition in the subject. The full therapeutic effect does not necessarily occur by administration of one dose and may occur only after administration of a series of doses. Thus, a therapeutically effective amount may be administered in one or more administrations.

The term “treating” a disease, disorder or condition in a patient, as used herein, refers to taking steps to obtain beneficial or desired results, including clinical results. For purposes of this invention, beneficial or desired clinical results include, but are not limited to alleviation or amelioration of one or more symptoms of the disease; diminishing the extent of disease; delaying or slowing disease progression; amelioration and palliation or stabilization of the disease state.

The term “vismodegib” as used herein means an FDA-approved Smoothened (Smo or SMO) inhibitor for treating advanced basal cell carcinoma (BCC).

Generally, nomenclature used in connection with, and techniques of, cell and tissue culture, molecular biology, immunology, microbiology, genetics, protein, and nucleic acid chemistry and hybridization described herein are those well-known and commonly used in the art. The methods and techniques of the present invention are generally performed according to conventional methods well known in the art and as described in various general and more specific references that are cited and discussed throughout the present specification unless otherwise indicated. See, e.g., Sambrook et al. Molecular Cloning: A Laboratory Manual, 2d ed., Cold Spring Harbor Laboratory Press, Cold Spring Harbor, N.Y. (1989); Ausubel et al., Current Protocols in Molecular Biology, Greene Publishing Associates (1992, and Supplements to 2002); Harlow and Lan, Antibodies: A Laboratory Manual, Cold Spring Harbor Laboratory Press, Cold Spring Harbor, N.Y. (1990); Principles of Neural Science, 4th ed., Eric R. Kandel, James H. Schwartz, Thomas M. Jessell editors. McGraw-Hill/Appleton & Lange: New York, N.Y. (2000). Unless defined otherwise, all technical and scientific terms used herein have the same meaning as commonly understood by one of ordinary skill in the art.

### 2. Background

Shh or sonic hedgehog pathways, herein also called hedgehog (Hh) pathways, are present in vertebrate development (1). Of the three mammalian Hh ligands, sonic hedgehog (Shh) regulates a wide range of biological activities, from establishing left-right body asymmetry and limb patterning to eye and central nervous system development. The other two, desert hedgehog (Dhh) and Indian hedgehog (Ihh), are mainly involved in the development of male germ cells and cartilage, respectively (1). PTCH1 is the Hh receptor of a 12-transmembrane domain protein. In the absence of Shh, PTCH1 blocks Hh signaling by repressing a membrane-bound G-protein-coupled receptor-like protein SMO (1, 2). The binding of Shh to PTCH1 relieves the repression of SMO, triggering a canonical Hh response whereby SMO moves to the primary cilium where it activates the GLI family of transcription factors. GLI forms a cytoplasmic complex with several accessory modulators, including the serine-threonine kinase Fused (FU), Suppressor of Fused (SuFU), and costal2 (COS2), a kinesin-related protein that binds the GLI-containing complex to microtubules. It is believed that SMO activity favors dissociation of these complexes and translocation of an active form of GLI from the cytoplasm to the nucleus, where it promotes the transcription of Hh target genes, including PTCH1, GLI, insulin-like growth factor 2 (IGF-2), cyclin D, bone morphogenic proteins (BMPs), and a member of the TGF-β superfamily (1-3).

Aberrant activation of the Shh pathway is responsible for many developmental and congenital disorders in humans and the development and/or maintenance of numerous types of human cancers, including cancers of the colon, lung, pancreas, and medulloblastoma. It also regulates the proliferation of cancer stem cells (CSCs) that are believed to drive tumor progression metastasis and may also hasten tumor relapse by augmenting multidrug resistance (MDR) pathways (2, 4, 5). Ligand-independent Hh pathway activation, due to loss-of-function mutations in PTCH1, gain-of-function mutations in SMO, and missense mutations in GLI1 and GLI3, underlies the development of basal cell carcinoma (BCC), the most common human malignancy in the US. In addition, the rare, dominantly inherited disorder known as nevoid basal cell carcinoma syndrome (BCCNS, also known as Gorlin syndrome) is caused by PTCH1 haplodeficiency (6). Individuals with BCCNS develop a few to thousands of BCCs, beginning as early as age two, and are at increased risk for medulloblastoma and rhabdomyosarcomas (7). The clinical usefulness of drug therapy targeted to cancer-related signaling pathways is illustrated by the results of our recent Phase 2 trial of the orally administered SMO inhibitor GDC-0449 (vismodegib) in BCCNS patients showing substantial efficacy and confirming that Hh/Shh signaling drives BCC tumorigenesis (8). However, substantial tumor recurrence after discontinuation of the drug treatment, together with the lack of apoptosis in regressed tumors, suggest the additional genetic factors and pathways are likely involved in the growth of BCCs.

The clinical usefulness of drug therapy targeted to cancer-related signaling pathways is illustrated by the results of our recent Phase 2 trial of the orally administered SMO inhibitor GDC-0449 (vismodegib) in BCCNS patients showing substantial efficacy and confirming that Hh signaling drives BCC tumorigenesis (8). However, substantial tumor recurrence after discontinuation of the drug treatment, together with the lack of apoptosis in regressed tumors, suggest the additional genetic factors and pathways are likely involved in the growth of BCCs.

Several lines of evidence demonstrate the interconnection of Shh signaling with other pathways: a high risk of BCCs is associated with cartilage-hair hypoplasia (CHH), an inherited disorder due to mutations in the RMRP (RNA component of Mitochondrial RNA-Processing endoribonuclease), and xeroderma pigmentosum (XP), an autosomal recessive disorder of DNA repair, implicating the involvement of non-canonical pathways in BCC tumorigenesis (9). A connection between the p53 tumor suppressor and Shh pathway has also been demonstrated, as the loss of p53 results in increased SMO expression in the interfollicular epidermis in mice (10). Because synergistic enhancement of oncogenic signaling can drive malignant transformation (11), defining mechanisms underlying pathway cross-talk are involved for the identification of novel druggable downstream targets in BCC (12).

Although germline Ptch 1 mutations in the Sonic hedgehog (Shh) signaling pathway drive the spontaneous growth of basal cell carcinomas (BCC) in Gorlin/basal cell nevus syndrome (BCNS), Shh-targeted therapies in these patients are still associated with tumor recurrence, coupled with the acquisition of drug-resistant secondary mutations in smoothened (SMO), which suggests that additional mechanisms underlie BCC carcinogenesis.

### 3. Overview

Sonic hedgehog (Shh)-targeted therapies, for example with vismodegib, in basal cell nevus syndrome (BCNS) patients have shown limitations including tumor recurrence, acquired drug resistance, and resistance to apoptosis in regressed tumors (Tang, 2012 #2857). Thus inhibition of Shh signaling alone is insufficient for permanent eradication of BCCs and alternate strategies are needed to enhance efficacy.

The Akt pathway regulates cell survival and apoptosis and is frequently dysregulated in numerous types of human cancers (13, 14). Akt1 is also aberrantly activated in cancers that develop in the skin (14, 15). However, neither the mechanisms whereby Akt is activated in BCCs nor the full spectrum of the oncogenic consequences of Akt activation in BCC tumorigenesis are known (16). The results here show that over-expression of constitutively active (myristoylated) Akt1/PKB increased colony formation and proliferation of normal human keratinocytes in vitro and inhibited UV-induced apoptosis, thereby conferring a selective survival advantage to these normal keratinocytes. It is now shown for the first time that activation of Akt1 (to the phosphorylated form Akt1 S473) is pivotal for the growth of BCCs shown in part by the fact that the partial genetic ablation of Akt1 effectively abrogated the formation of BCCs in Ptch1+/31 /SKH-1 mice. Akt1 is directly regulated by the Shh pathway transcription factor SOX9 via mTOR.

By screening a panel of Akt inhibitors currently in active clinical trials for various other human cancers, it was discovered that, while Akt1 inhibition significantly reduced Akt phosphorylation and proliferation of cultured BCC (ASZ001) cells to varying degrees, as assessed by BrdU incorporation, only the alkyl-lysophospholipid analogs perifosine and edelfosine further induced apoptosis in BCC in addition to reducing proliferation, as shown by an increase in cleaved caspase-3. When the combination of perifosine with an SMO inhibitor (itraconazole or vismodegib) was tested, a synergistic reduction in cell proliferation in vitro was achieved. Importantly, in vivo treatment of animals with BCC with the combination of perifosine and either itraconazole or vismodegib, produced a synergistic reduction of tumor burden and the number of BCCs in chronically UV-irradiated Ptch+/−SKH-1 mice.

The results presented here delineate the mechanism whereby aberrant Shh signaling converges on the Akt1-mTOR pathway, conferring selective growth advantage and enhanced survival of BCC tumor cells. Utilizing a mouse model of BCNS made in our lab (Chaudhary S C, In press #2833), a pivotal role has been discovered for Akt1 signaling in BCC tumorigenesis. The results support embodiments for treating BCC/BCNS and other cancers as described below.

### 4. Embodiments

Using Shh inhibitors for Shh+ tumors is not new, nor is using Akt/mTOR inhibitors for Akt+ and/or mTOR+ tumors. However, the discovery that Shh pathway activation directly regulates mTOR-Akt means that Akt inhibitors such as perifosine alone and mTOR inhibitors alone, can be used to treat BCC. Combination therapy with Akt plus Shh inhibitors is even more effective in treating BCC, since it can inhibit mTOR-independent Akt targets. BCC can also be treated by adding an mTOR inhibitor to Akt inhibitor, or by administering a combination of Akt plus Shh plus mTIR inhibitors. The results also support embodiments for therapies targeting other tumors besides BCC that are active for both Akt and Shh with combination therapy using an Akt inhibitor such as perifosine and an Shh inhibitor. Other embodiments are directed to methods of treating cancers classified as Shh activated (Shh+) by combining treatment with an SPI inhibitor such as a SMO inhibitor with an Akt inhibitor and/or mTOR inhibitor. This is because such Shh+ cancers can also be Akt+. In addition to various embodiments described herein of therapeutic methods for treating Akt+ cancers, and Shh+/Akt+ cancers, Shh+/mTOR+ cancers and other embodiments are directed to a pharmaceutical formulations comprising (1) a therapeutically effective amount of an Akt1 inhibitor together with a sonic hedgehog activated pathway inhibitor. In some embodiments the Akt inhibitor can be perifosine, edelfosine, MK-2206, AZD5363, pharmaceutically active derivatives thereof or combinations thereof. In some embodiments the mTOR inhibitor is rapamycin, Agent OSI-027, XL765, everolimius, temsirolimus, zotarolimus, pharmaceutically active derivatives thereof, or combinations thereof. Certain SMO inhibitors in addition to vismodegib and itraconazole for use in embodiments of the invention are listed in Tables 1 and 2.

Other embodiments are directed to a pharmaceutical formulations comprising (1) a therapeutically effective amount of an Akt1 inhibitor together with a sonic hedgehog activated pathway inhibitor, (2) a therapeutically effective amount of an Akt1 inhibitor together with an mTOR inhibitor, or (3) combinations of an Akt1 inhibitor together with a sonic hedgehog activated pathway inhibitor and an mTOR inhibitor. Sonic hedgehog pathway inhibitors include smoothened inhibitors such as vismodegib and itraconazole or pharmaceutically active derivatives thereof.

In some embodiments for treating skin cancers like sporadic BCC in the general population, BCC in BCNS patients, and BCCs in XP patients, the composition is formulated for topical or oral administration. Embodiments of the pharmaceutical compositions can be formulated in any way known in the art to optimize delivery to and treatment of the cancers described here, such as for injection.

### 5. Pharmaceutical Compositions and Formulations

Certain embodiments of the present invention are directed to pharmaceutical compositions and formulations of the active agents as described herein for treatment of BCC, and other cancers that involve activation of the Shh pathway, including colon, pancreatic, medulloblastoma, prostatic, esophageal, glioma, and gastrointestinal cancers, collectively “the enumerated diseases.” The active agents include Akt1 inhibitors, mTOR inhibitors and Shh pathway inhibitors (SPI) including SMO inhibitors as described herein and others known to persons of skill in the art.

The therapeutic agents are generally administered in an amount sufficient to treat or prevent an enumerated disease. The pharmaceutical compositions of the invention provide a therapeutic amount of the active agents effective to treat or prevent an enumerated disease or disorder. In certain embodiments, the pharmaceutical compositions of the present invention comprise about 0.1 mg to 5 g of each active agent. The therapeutic dose can vary widely for example from about 1-25 mg/day, 25-50 mg/day, 50-100 mg/day, 100-200 mg/day, 200-300 mg/day, 400-500 mg/day and 500-1000 mg/day, 0.5 mg to about 1 g, about 1 mg to about 750 mg, about 5 mg to about 500 mg, or about 10 mg to about 100 mg of therapeutic agent. On the basis of a previous phase I study evaluating the safety and maximum tolerated dose (MTD) of perifosine in renal cell carcinoma, a dose of about 1 mg/day to 500 mg/day of Akt inhibitor, SPI including SMO inhibitors and mTOR inhibitors is suggested. Schreeder MTFRA, Stephenson J J, Campos L, Chawla S P, Spigel D R, Spira A, et al. Phase I multicenter trial of perifosine in combination with sorafenib for patients with advanced cancers including renal cell carcinoma. J Clin Oncol 2008, 20 (suppl; abstr 16024).

It is understood that the appropriate dose of an active agent depends upon a number of factors within the ken of the ordinarily skilled physician, veterinarian, or researcher for example, the identity, size, and condition of the subject or sample being treated, further depending upon the route by which the composition is to be administered, the frequency of administration, the severity of the disease, and the effect which the practitioner desires the an active agent to have. It is furthermore understood that appropriate doses of an active agent depend upon the potency with respect to the expression or activity to be modulated. Such appropriate doses may be determined using the assays described herein. When one or more of these active agents are to be administered to an animal (e.g., a human), a relatively low dose may be prescribed at first, with the dose subsequently increased until an appropriate response is obtained. In addition, it is understood that the specific dose level for any particular subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

Active agents can be administered as a single treatment or, preferably, can include a series of treatments that continue at a frequency and for duration of time that causes one or more symptoms of the enumerated disease to be reduced or ameliorated, or that achieves the desired effect including reducing tumor burden or metastasis. Typical frequencies of administration of therapeutic agents in embodiments of the invention include once per day, multiple times per day, every few days, every week or every few weeks, as needed and as determined by the physician. Active agents administered “together” can be administered at the same time in the same or different formulations, or at different times.

Active agents of the invention may be chemically modified to facilitate uptake by the skin, for BCC, or by the brain or pancreas or other target organ using methods known in the art.

The term “administer” is used in its broadest sense and includes any method of introducing the compositions of the present invention into a subject. Administration of an agent “in combination with” includes parallel administration of two agents to the patient over a period of time, co-administration (in which the agents are administered at approximately the same time, e.g., within about a few minutes to a few hours of one another), and co-formulation (in which the agents are combined or compounded into a single dosage form suitable for oral, topical, subcutaneous or parenteral administration).

The pharmaceutical compositions of the present invention may be administered in a number of ways depending upon whether local (to the skin or tumors) or systemic treatment is desired and upon the area to be treated. For BCC topical administration may be preferred, which also means that higher doses can be applied that might be administered systemically. Topical and systemic administration are not mutually exclusive.

Administration can also be intravenous, parenteral, intra-arterial, subcutaneous, intraperitoneal or intramuscular injection or infusion; or intracranial, e.g., intrathecal or intraventricular, administration. In recent years there has been a tendency towards the development of controlled release dosage forms that will provide therapy over an extended period of time. Normally this would be once a day, and it is believed that such a change in dosage regimen will reduce adverse reactions and side effects and also improve patient compliance. The use of synthetic polymers that may have muco- or bio-adhesive properties has been investigated and is disclosed in WO 85/02092.

In some embodiments, a slow-release preparation comprising the active agents is formulated. It is desirable to prolong delivery with these slow release preparations so that the drug may be released at a desired rate over this prolonged period. By extending the period, the drug can if required be released more slowly, which may lead to less-severe adverse reactions and side effects. The preparation of sustained, controlled, delayed or anyhow modified release form can be carried out according to different known techniques: 1. The use of inert matrices, in which the main component of the matrix structure opposes some resistance to the penetration of the solvent due to the poor affinity towards aqueous fluids; such property being known as lipophilia; 2. The use of hydrophilic matrices, in which the main component of the matrix structure opposes high resistance to the progress of the solvent, in that the presence of strongly hydrophilic groups in its chain, mainly branched, remarkably increases viscosity inside the hydrated layer; and 3. The use of bioerodible matrices, which are capable of being degraded by the enzymes of some biological compartment. See U.S. Pat. No. 7,431,943.

The term “slow release” refers to the release of a drug from a polymeric drug delivery system over a period of time that is more than one day wherein the active agent is formulated in a polymeric drug delivery system that releases effective concentrations of the drug. Drug delivery systems may include a plurality of polymer particles containing active drug material, each of the particles preferably having a size of 20 microns or less, and incorporating on the outer surface of at least some of the particles a bioadhesive material derived from a bacterium. Such drug delivery systems have been described in U.S. Pat. No. 6,355,276. The use of these microorganisms in the design allow for a controlled release dosage form with extended gastrointestinal residence.

In certain embodiments, dosage forms of the compositions of the present invention include, but are not limited to, implantable depot systems.

Self emulsifying microemulsion drug delivery systems (SMEDDS) are known in the art See U.S. Patent Application 2001/00273803. The term SMEDDS is defined as isotropic mixtures of oil, surfactant, cosurfactant and drug that rapidly form oil in water microemulsion when exposed to aqueous media or gastrointestinal fluid under conditions of gentle agitation or digestive motility that would be encountered in the GI tract.

Thermostable nanoparticles may be contained in a drug delivery system targeted for the GI tract. See U.S. Patent Application 2000/60193787. These drug delivery systems may include at least one type of biodegradable and/or bioresorbable nanoparticle and at least one drug that possesses at least one of the following properties: emulsifier or mucoadhesion. The drug may substantially cover the surface of the nanoparticle.

The therapeutic agent can be formulated with an acceptable carrier using methods well known in the art. The actual amount of therapeutic agent will necessarily vary according to the particular formulation, route of administration, and dosage of the pharmaceutical composition, the specific nature of the condition to be treated, and possibly the individual subject. The dosage for the pharmaceutical compositions of the present invention can range broadly depending upon the desired effects, the therapeutic indication, and the route of administration, regime, and purity and activity of the composition.

A suitable subject, preferably a human, can be an individual or animal that is suspected of having, has been diagnosed as having, or is at risk of developing an enumerated disease, and like conditions as can be determined by one knowledgeable in the art.

Techniques for formulation and administration can be found in “Remington: The Science and Practice of Pharmacy” (20th edition, Gennaro (ed.) and Gennaro, Lippincott, Williams & Wilkins, 2000), incorporated herein by reference.

The pharmaceutical compositions of the present invention may be administered in a number of ways depending upon whether local or systemic treatment is desired and upon the area to be treated. Administration may be-oral or parenteral. Parenteral administration includes intravenous, intraarterial, subcutaneous, intraperitoneal or intramuscular injection or infusion; or intracranial, e.g., intrathecal or intraventricular, administration.

Active agents may be admixed, encapsulated, conjugated or otherwise associated with other molecules, molecule structures or mixtures of compounds, as for example, liposomes, receptor targeted molecules, oral, rectal, topical or other formulations, for assisting in uptake, distribution and/or absorption. Representative United States patents that teach the preparation of such uptake, distribution and/or absorption assisting formulations include, but are not limited to, U.S. Pat. Nos. 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756, each of which is herein incorporated by reference.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylene diamine tetra acetic acid; buffers such as acetates, citrates or phosphates; and agents for the adjustment of tonicity such as sodium chloride or dextrose. pH can be adjusted with acids or bases, such as hydrochloric acid or sodium hydroxide. The parenteral preparation can be enclosed in ampules, disposable syringes or multiple dose vials made of glass or plastic.

Pharmaceutical compositions suitable for injectable use include sterile aqueous solutions (where the therapeutic agents are water soluble) or dispersions and sterile powders for the extemporaneous preparation of sterile injectable solutions or dispersion. For intravenous administration, suitable carriers include physiological saline, bacteriostatic water, Cremophor EL® (BASF, Parsippany, N.J.) or phosphate buffered saline (PBS). In all cases, the composition must be sterile and should be fluid to the extent that easy syringability exists. It should be stable under the conditions of manufacture and storage and should be preserved against the contaminating action of microorganisms such as bacteria and fungi.

The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active agent in the required amount in an appropriate solvent with one or a combination of the ingredients enumerated above, as required, followed by filter sterilization. Generally, dispersions are prepared by incorporating the active agent into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. Depending on the specific conditions being treated, pharmaceutical compositions of the present invention for treatment of atherosclerosis or the other elements of metabolic syndrome can be formulated and administered systemically or locally. Techniques for formulation and administration can be found in “Remington: The Science and Practice of Pharmacy” (20th edition, Gennaro (ed.) and Gennaro, Lippincott, Williams & Wilkins, 2000). For oral administration, the agent can be contained in enteric forms to survive the stomach or further coated or mixed to be released in a particular region of the GI tract by known methods. For the purpose of oral therapeutic administration, the active agent can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, PRIMOGEL® or corn starch; a lubricant such as magnesium stearate or STEROTES® a glidant such as colloidal silicon dioxide; a sweetening agent such as sucrose or saccharin; or a flavoring agent such as peppermint, methyl salicylate, or orange flavoring.

Systemic administration can also be by transmucosal means to the intestinal or colon. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active agents are formulated into ointments, salves, gels, or creams as generally known in the art.

In one embodiment, the active agents are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to particular cells with, e.g., monoclonal antibodies) can also be used as pharmaceutically acceptable carriers. These can be prepared according to methods known to those skilled in the art, for example, as described in U.S. Pat. No. 4,522,811.

### 6. Summary of Experimental Results

The Ptch1^{+/-}/SKH-1 mice animal model used in experiments described here closely recapitulates the growth pattern of BCCs in humans, particularly the accelerated growth of these tumors in patients with BCNS (Chaudhary S C, In press #2833). Animals were made by introducing Ptch1^{+/-} onto the SKH-1 hairless background, to generate Ptch1^{+/-}/SKH-1 mice that are uniquely susceptible to the development of numerous spontaneous and UV-induced BCCs (Chaudhary S C, In press #2833). It was also discovered that mTOR, which phosphorylates Akt at S473, was elevated both in human BCCs and in BCCs that develop in Ptch1^{+/-}/SKH-1 mice. mTOR mRNA and Akt S473 phosphorylation were constitutively increased in keratinocytes isolated from neonatal Ptch1^{+/-}/SKH-1 mice, indicating that mTOR-Akt1 functions downstream of the Shh pathway. mTOR was identified as a direct transcriptional target of Shh signaling.

The promoter region of mTOR possesses binding motifs for SOX9, a GLI-regulated transcription factor in the Shh pathway. Both endogenous and recombinant SOX9 proteins directly bind to the mTOR promoter in Ptch1^{+/-}/SKH-1 mice in vivo. Genetic ablation of SOX9 in BCC cells diminished its binding to mTOR promoter thereby inhibiting expression of mTOR, resulting in a blockade of phosphorylation of well-characterized mTOR substrates including p7056K and 4E-BP1. Importantly, it was discovered that both Akt1 haplodeficiency and pharmacological inhibition of Akt with the Akt1 inhibitor perifosine significantly reduced the growth of both spontaneous and UV-induced BCCs in Ptch1⁺⁻/SKH-1 mice. The Akt isoforms (Akt1, Akt2 and Akt3) share structural homology in mammals including humans, but display distinct physiological roles as well as tissue specificity [42]. Akt1 and Akt2, but not Akt3, are expressed in skin. Akt1 has been the best-studied isoform relating to the pathogenesis of skin cancer. Our analysis of Akt1+/−/Ptch1+/−/SKH-1 mice show that Akt2 levels are unchanged as compared with Ptch1+/−/SKH-1 mice. Thus, Shh signaling drives Akt1 via the SOX9-mTOR regulatory axis showing that Akt1 is a mechanism-driven target for pharmacological inhibition of BCC pathogenesis in patients with BCNS.

UVB radiation is a complete carcinogen, and UVB exposure is a known major risk factor for the induction of BCCs, both in the general population and in BCNS patients. Sun-exposed areas develop more BCCs compared to sun-protected areas. In Ptch1+/−/SKH-1 mice, UVB irradiation greatly enhances the number and size of BCCs. Furthermore, Akt1 phosphorylation is increased in UVB-irradiated BCCs compared to spontaneous BCCs suggesting that Akt1 activation may be involved in the growth of both spontaneous and UVB-induced BCCs.

Akt1 is activated in ASZ001 BCC cells, originally derived from a murine BCC [44]. Treatment of ASZ001 cells with LY294002, a PI3K inhibitor, and cyclopamine, a SMO inhibitor, synergistically decreased cell proliferation as evidenced by a substantial reduction of BrdU incorporation into the DNA of these cells and abrogation of cyclin D1 expression. Conversely, constitutive activation of the Akt and Shh pathways via expression of myristoylated Akt1 (myr-Akt1) [45] and Gli2ΔN [46] (removal of the amino-terminal repressor domain results in potent transcriptional activity), respectively, synergistically increased BrdU incorporation in primary keratinocytes, indicating synergistic enhancement of proliferative capacity following co-activation of the Shh and Akt pathways.

An experiment was designed to test the feasibility of inhibiting Shh and PI3K/Akt pathways in vivo, either alone or in combination, to evaluate the effect on the growth of existing BCCs in Ptch1+/−/SKH-1 mice. Ptch1+/−/SKH-1 mice were irradiated with UVB to induce BCCs, and the mice were then treated with either SMO inhibitor cyclopamine and P13K inhibitor LY294002 or a combination of the two for four weeks. Inhibition of both SMO and Akt1 signaling led to a substantial decrease in the growth of UVB-induced BCCs.

The following is a summary of results of experiments described in the Examples of this application:
Shh inhibition (itraconazole or vismodegib) suppressed Akt1-mTOR signaling in vitro and in vivo;
Genetic ablation of SOX9 reduced the expression of mTOR and the phosphorylation of p-p-7056K, p-4E-BP1 and cyclin D1 in ASZ001 cells to a level comparable to the inhibition of mTOR signaling by rapamycin;
Reduction in mTOR expression following pharmacological inhibition of the Shh pathway or SOX9 knockdown suggested that Shh signaling transcriptionally regulates mTOR proving that mTOR is a direct transcriptional downstream target of SOX9;
Assessment of the levels of Akt1 Ser473 phosphorylation in primary keratinocytes isolated from newborn Ptch 1+/−/SKH-1 mice showed that Akt1 is intrinsically activated in Ptch1^{+/-} postnatal keratinocytes;
Analysis of Akt1+/−/Ptch1+/−/SKH-1 mice showed that Akt2 levels were unchanged as compared to Ptch1+/−/SKH-1 mice and Akt1 haplodeficiency prevented the formation of spontaneous BCCs in Ptch1^{+/-}/SKH-1 mice;
p-Akt1 S473 co-localized with Gli1 or SOX9 in BCC cells and genetic inhibition of Akt1 in BCC cells substantially reduced the development of UV-induced BCCs in Ptch1+/−/SKH-1 mice, confirming the integral role of Akt1 activation in BCC pathogenesis and that Akt1 haplodeficiency prevents UV-induced BCC development in Ptch1^{+/-}/SKH-1 mice; and
Akt signaling in BCCs treated with the SMO inhibitor cyclopamine and P13K inhibitor LY294002 resulted not only in a reduction in mTOR (phosphorylated mTOR) levels, but also in a reduction in total mTOR suggesting that mTOR is transcriptionally regulated. Akt signaling was inhibited pharmacologically in ASZ001 cells using Akt inhibitors and oral administration of perifosine was effective in preventing UV-induced BCC development.
In both ASZ001 and myrAkt1-transformed ASZ001 cells, treatment regimens with the Akt inhibitor perifosine and either SMO inhibitor vismodegib or itraconazole, performed better than either drug alone.
In vivo treatment of BCC in Ptch1+/−/SKH-1 mice showed perifosine combined with SMO inhibitors acts synergistically.

### 7. EXAMPLES

The invention is illustrated herein by the experiments described by the following examples, which should not be construed as limiting. The contents of all references, pending patent applications and published patents, cited throughout this application are hereby expressly incorporated by reference. Those skilled in the art will understand that this invention may be embodied in many different forms and should not be construed as limited to the embodiments set forth herein. Rather, these embodiments are provided so that this disclosure will fully convey the invention to those skilled in the art. Many modifications and other embodiments of the invention will come to mind in one skilled in the art to which this invention pertains having the benefit of the teachings presented in the foregoing description. Although specific terms are employed, they are used as in the art unless otherwise indicated.

### Example 1

### Materials and Methods

### Cells and Reagents

Adult normal human epidermal keratinocytes (Lonza, Switzerland) and murine ASZ001 BCC cells (gift from Dr. Ervin Epstein) were cultured in KGM-gold keratinocyte growth medium (Lonza, Switzerland). pUSEamp-myrAkt1 was obtained from Millipore (Billerica, Mass.). ASZ001 cells and normal human keratinocytes were transfected with pUSEamp-myrAkt1 at 10 μg using lipofectamine 2000 (Invitrogen, Carlsbad, Calif.) and stable colonies were selected in the presence of G418 (Sigma-Aldrich, St Louis, Mo.).

### Western Blotting and Immunohistochemical Analyses

Western blotting and immunohistochemical analyses were performed as previously described (50) (51). Antibodies against Akt, p-Akt1, Akt2, Akt3, p-Akt, GLI1, GLI2, mTOR, p-p70S6K, p-4E-BP1, and 4E-BP1 were from Cell Signaling Technology (Danvers, Mass.), cyclin D1 from Santa Cruz Biotechnology (Santa Cruz, Calif.), SOX9 from Abcam (Cambridge, Mass.), and β-actin from Sigma-Aldrich (St. Louis, Mo.). Perifosine and AZD5363 were purchased from Selleckchem (Houston, Tex.), edelfosine from Sigma-Aldrich (St. Louis, Mo.), and MK-2206 from ChemieTek (Indianapolis, Ind.).

### TUNEL Assay

Primary human keratinocytes were transduced with the viral construct expressing myrAkt1 (pUSEamp-myrAkt1) or pUSEamp vector (Millipore, Billerica, Mass.). Virally transduced cells were then irradiated with UV (30 mJ/cm2). The TUNEL assay was performed using an in situ cell death detection kit, fluorescein (Roche Applied Science). DNA fragmentation was detected using Tecan GENios Plus system (Mannedorf, Switzerland) according to the manufacturer's instructions.

### Colony Formation Assay to Determine Anchorage-Independent Cell Growth

pBAP-myrAkt1-transduced primary human keratinocytes were irradiated with UV (30 mJ/cm2). Cells were then resuspended in top agar (0.7% agarose in keratinocyte media) and placed with the base layer prepared in 0.8% agar in keratinocyte media with supplements. After 3 weeks, cells were stained with Crystal Violet and the number of colonies counted. The experiment was repeated twice independently. Statistical analysis was performed with Student's t-test. P<0.05 was considered statistically significant.

shSOX9 Knockdown in ASZ001 Cells

Murine shSOX9 were purchased from OriGene (Rockville, Md.). Construction of stable SOX9 knockdown cell lines (shSOX9-ASZ001) was performed according to the guidelines of the Phoenix retroviral expression system (Orbigen Inc., San Diego, Calif.).

### Electrophoretic Mobility Shift (EMSA) and Supershift Assays Using Recombinant MBP-SOX9

Nuclear extracts from shSOX9-ASZ001 cells and shGFP-ASZ001 cells were prepared using a Nuclear Extraction Kit (Pierce Biotech., Rockford, Ill.). SOX9 binding activity was determined using 20 μg of nuclear proteins with 1 pmol of ³²P-labeled mTOR promoter consensus oligonucleotides; pmTOR4069SOX9BE (5′-TCAATGGTTTTTTGCTTTGTTTCT GGAGACAGGGCC-3′) (SEQ ID NO: 1); pmTOR9313 SOX9BE (5′-AGGCTA ATTCTGAACAATGGTGAGTTCTGTT-3′) (SEQ ID NO: 2); pmTOR15126 SOX9BE (5′-GGAAAGCAAAGAAACAAAGCCACTGACATAC-3′) (SEQ ID NO: 3). A 100-fold excess of unlabeled oligonucleotide was added for competition reaction for 20 min, prior to the addition of ³²P-labeled mTOR promoter consensus oligonucleotides, to confirm the specificity of SOX9 binding. The protein-DNA complexes were separated on 5% Tris/Boric Acid/EDTA (TBE) gel and bands detected by autoradiography on X-ray film (Kodak, Rochester, N.Y.). Recombinant SOX9 protein fused to maltose-binding protein (MBP) (MBP-50X9) was purified using the pMAL purification system (New England Biolabs, Ipswich, Mass.). For supershift, 2 μl of Anti-MBP antibody (New England Biolabs, Ipswich, Mass.) were added to the reaction mixture.

### Chromatin Immunoprecipitation (ChIP) Assay

ChIP assay was performed using SimpleChIP™ Enzymatic Chromatin IP kit (Cell signaling Tech., Danvers, Mass.) according to the manufacturer's instructions with minor modifications. In brief, DNA and proteins were cross-linked with formaldehyde for 10 min at room temperature; cross-linking was terminated by the addition of glycine at a final concentration 0.125 mM. Cells were harvested by scraping, resuspended in lysis buffer, and incubated on ice for 10 min. A lysis mixture was centrifuged for 5 min at 1500 rpm to remove supernatant. Pellets were resuspended in buffer containing micrococcal nuclease and incubated for 20 min at 37° C. to digest chromosomes. The reaction was stopped by adding 0.5 M EDTA. Nuclei pellets were resuspended in ChIP buffer, DNA sheared on ice using a Misonix Sonicator 3000 (Misonix, Farmingdale, N.Y.) and resolved on agarose gels to confirm that the average fragment sizes were between 150 and 900 bp. 20 μl of the sonicated samples were used for total DNA loading controls. DNA concentrations were determined, and equal amounts of chromatin were used for further processing. Samples were diluted with ChIP dilution buffer and chromatin was incubated with 2 mg of the appropriate antibody (i.e., anti-SOX9 (H-90) (Santa Cruz Biotechnology, Santa Cruz, Calif.); anti-Sox9 (Abcam, Cambridge, Mass.); and anti-Histone H3 (Cell Signaling, Danvers, Mass.)) at 4° C. for ˜12-16 h. Chromatin-antibody complexes were washed and eluted and cross-links reversed by incubating at 65° C. for 30 min. The samples were then treated with proteinase K at 65° C. for 2 h. DNA was recovered using a DNA purification kit. PCR was performed using region-specific primers mTOR9313IPF (5′-GATGGAAAGGGATGAGCAGA-3′) (SEQ ID NO: 4) and mTOR9313IPR (5′-CCAGGATCGAACTCTCCAAC-3′) (SEQ ID NO: 5), mTOR4069IPF (5′-GCCAAGAGCTTGGAAAAGAA-3′) (SEQ ID NO: 6) and mTOR4069IPR (5′-CTCTTGGGGGTGCTAGGAAT-3′) (SEQ ID NO: 7). All PCR products were resolved and visualized on agarose gels.

mTOR Promoter Assay

The promoter region of mTOR ranging 1 kb (pmTOR-1kb) and 2 kb (pmTOR-2kb) were cloned into pGL3-Basic vector (Promega, Madison, Wis.) at the KpnI and SacI sites, respectively. The pGL3-Basic-pmTOR-2kb plasmid was then used as the backbone for the construction of other plasmids. For pGL3-Basic-pmTOR-2kb-4069SOX9BE and pGL3-Basic-pmTOR-2kb-9313SOX9BE, putative SOX9 response element, the 4069SOX9BE and the 9313SOX9BE were inserted into the pGL3-Basic-pmTOR-2kb at the MluI and BglII sites, respectively. The integrated sequences were confirmed by sequencing. At 48 h after transfection, the luciferase reporter activity was measured using a luciferase assay system (Promega, Madison, Wis.).

Generation of Akt1^{+/-}/Ptch1^{+/-}/SKH-1 Mice and Assessment of Microscopic Lesions

Akt1^{+/-}/Ptch1^{+/-}/SKH-1 mice were generated by crossing heterozygous B6.129P2-Akt1tm1Mbb/J mice (Jacksons Lab) with Ptch1^{+/-}/SKH-1 mice. The resulting haired Akt1^{+/-}/Ptch1^{+/-} F1 littermates were backcrossed for 10 generations to generate Akt1^{+/-}/Ptch1^{+/-}/SKH-1. Animal experiments were performed in accordance with guidelines of our approved Columbia University Institutional Animal Care and Use Committee (IACUC) protocol. Akt1^{+/-}/Ptch1^{+/-}/SKH-1 and Ptch1^{+/-}/SKH-1 were sacrificed at 12 months of age. Skin samples were prepared using 10% buffered formalin-fixed H&E-stained and β-gal-stained samples of full-thickness dorsal skin. For β-gal staining, glutaraldehyde and formalin-fixed tissue were treated with X-gal and iron buffer solution (Roche Diagnostics Corp., Indiana) for 48 hours and processed using the vendor's protocol. Microscopic BCC-like lesions were defined as tumor islands composed of monomorphic basaloid cells with scant cytoplasm arranged as nests within the dermis. These lesions were counted as numbers per unit area and also as total tumor area per square millimeter skin section as previously described (52). For each mouse, three skin strips (avg. 1.5 cm×0.1 cm) were analyzed.

### UV Light Source

A UV Irradiation Unit (Daavlin Co., Bryan, Ohio) equipped with an electronic controller to regulate dosage was used. The UV source consisted of eight FS72T12-UV-HO lamps that emit UV (290-320 nm, 75-80% of total energy) and UVA (320-380 nm, 20-25% of total energy). A Kodacel cellulose film (Kodacel TA401/407) to eliminate UVC radiation was used. A UVC sensor (Oriel's Goldilux UVC Probe) was routinely used during each exposure to confirm lack of UVC emission. The dose of UV was quantified with a UV Spectrum 305 Dosimeter obtained from the Daavlin Co. (Bryan, Ohio). The radiation was further calibrated with an IL1700 Research Radiometer/Photometer from International Light Inc. (Newburyport, Mass.).

Tumor Study in Ptch1^{+/-}/SKH-1 and Akt1^{+/-}/Ptch1^{+/-}/SKH-1 Mice

UV study: Ptch1^{+/-}/SKH-1 (n=6) and Akt1^{+/-}/Ptch1^{+/-}/SKH-1 (n=5) and their wild type Ptch1^{+/+} littermates (WT, n=5) were irradiated with UV (180 mJ/cm² twice per week for 30 weeks). Itraconazole study: Ptch1^{+/-}/SKH-1 mice were UV-irradiated (180 mJ/cm² twice per week for 30 weeks) to induce BCCs. Irradiation was terminated. Mice were then treated with 2-hydroxypropyl-b-cyclodextrin or itraconazole (Sigma-Aldrich, St. Louis, Mo.) (twice daily, 100 mg/kg, i.p.) for 24 days. The size of tumors were measured as previously described. For the perifosine study, control group (n=4) received 0.9% NaCl/100 μl (gavage), while the experimental group (n=6) received perifosine (125 mg/kg body weight prepared in 0.9% NaCl, gavage, twice a week) 30 minutes prior to each UV irradiation. All animals were irradiated with 180 mJ/cm² UV twice weekly for 40 weeks. Tumor number and tumor volume were recorded once a week and plotted in terms of weeks on test.

### Statistical Analyses

Statistical analyses were performed using the Student's t test (two-tailed): p<0.05 was considered statistically significant.

### Example 2

### Akt1 is Activated in BCCs and Fosters the Survival of Human Keratinocytes

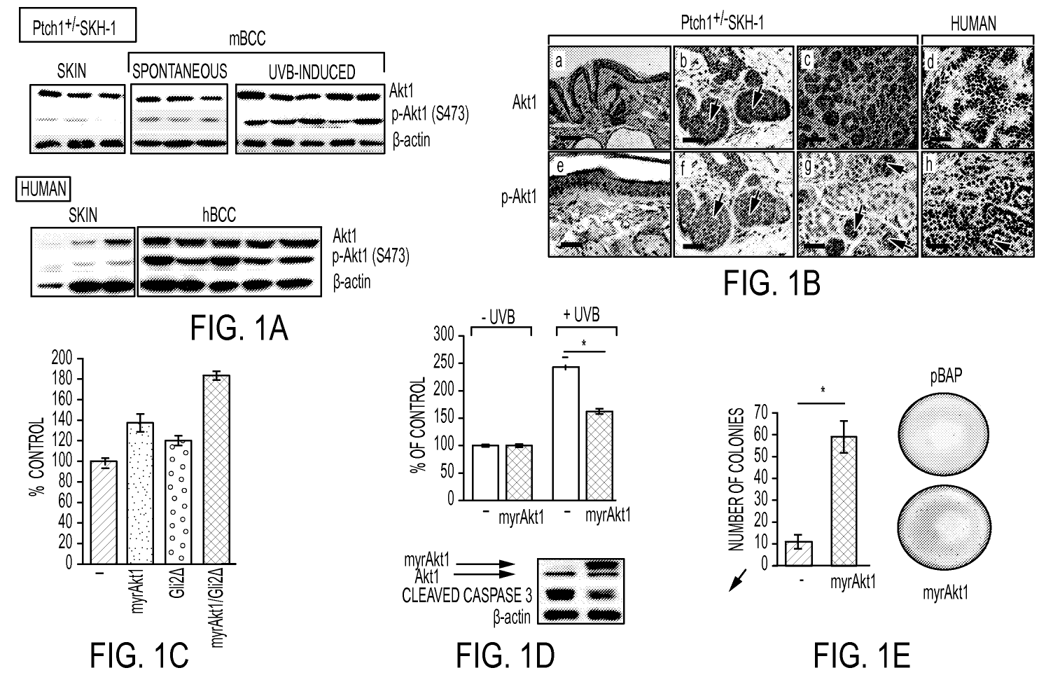

To determine the involvement of Akt1 in BCC pathogenesis, the extent of its S473 phosphorylation in Ptch1^{+/-}/SKH-1 mice was assessed, which is involved in the full activation of the PI3K-Akt pathway (17), In human BCCs (hBCC) and murine BCCs (mBCC). The levels of S473 Akt1 phosphorylation were increased in both spontaneous and UV-induced mBCCs as well as hBCCs compared to normal skin (FIG. 1A). Akt1 phosphorylation was limited to tumor cells and undetectable in tumor stroma (FIG. 1B, FIG. 1F, FIG. 1G, FIG. 1H). An increase in S473 phosphorylation correlated with the expression of the Shh signaling components, GLI1 and GLI2, in both human and murine BCCs (data not shown), indicating concurrent activation of both pathways in these tumors. Consistent with its role in cell survival and proliferation (13), constitutive activation of the Akt1 via expression of myristoylated Akt1 (myr-Akt1) (18) enhanced BrdU incorporation in normal human primary keratinocytes to a level comparable to that which resulted from activation of the Shh pathway by GLI2ΔN, a constitutive transcriptional-activator form of GLI2 (19) (FIG. 1C). Combined expression of myr-Akt1 and GLI2ΔN synergistically increased BrdU incorporation (FIG. 1C). Moreover, expression of myr-Akt1 protected keratinocytes from UV-induced apoptosis at a UV dose that totally killed vector-transduced control cells (FIG. 1D) and increased colony formation (FIG. 1E).

### Example 3

### Shh Inhibition Suppresses Akt1-mTOR Signaling in vitro and in vivo

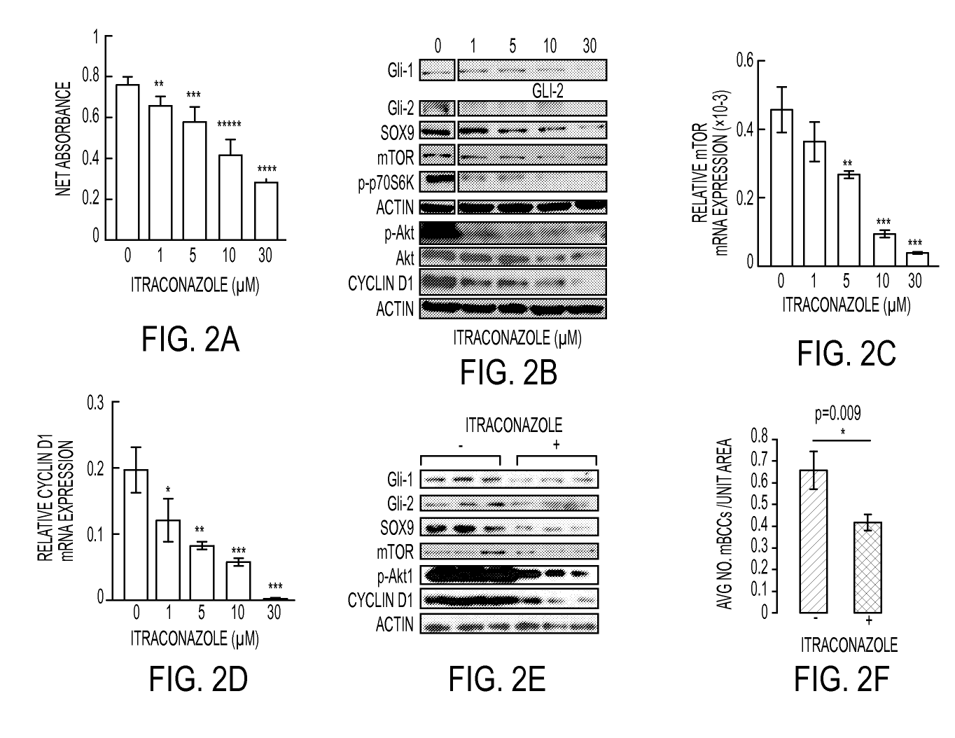

Itraconazole is an FDA-approved azole antifungal drug, recently shown to be a potent and specific inhibitor of Shh signaling (20). Treatment of ASZ001 cells (derived from BCCs induced in Ptch1^{+/-}/C57BL6 mice) (21) with itraconazole (1-30 μM), dose-dependently inhibited the growth of these cells (FIG. 2A) and decreased the levels of the Shh components, GLI1, GLI2, SOX9 (FIG. 2B), and cyclin D1 (FIGS. 2B, 2D). Interestingly, the administration of itraconazole also resulted in decreases in the total levels of mTOR, both proteins and mRNAs (FIGS. 2B, C), and resulted in a subsequent decrease in the phosphorylation of mTOR substrates, p70S6K and Akt (FIG. 2B). Oral administration of itraconazole suppressed Shh and Akt1-mTOR signaling in these mice (FIG. 2E) and the growth of existing UV-induced microscopic BCCs in Ptch1+/−/SKH-1 mice (FIG. 2F).

### Example 4

### SOX9 Knockdown Decreases mTOR Signaling

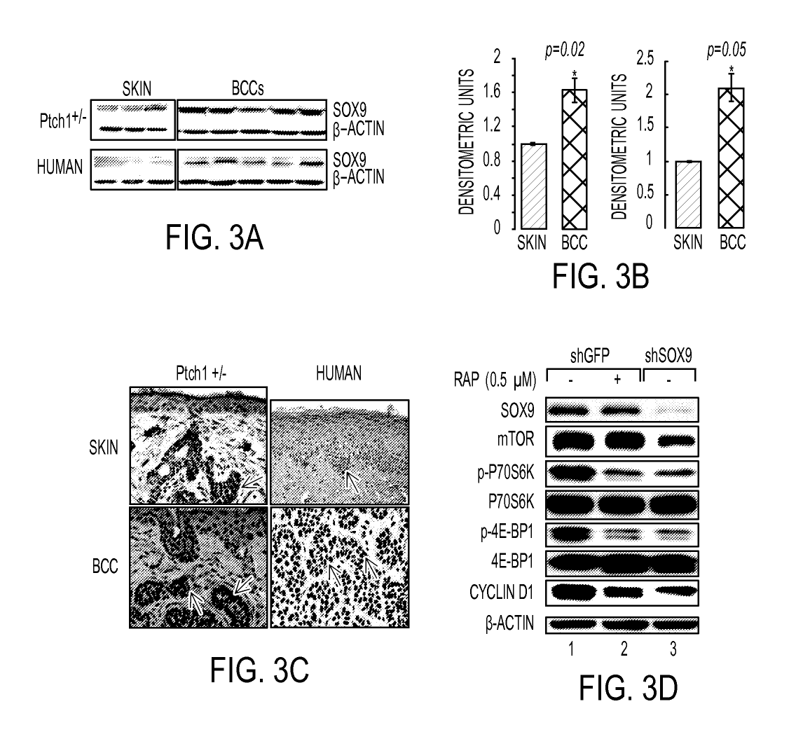

SOX9 belongs to group E of the SOX transcription factor family (SOX8, SOX9, and SOX10) defined by a common HMG box domain originally identified in SRY, the sex-determining gene on the Y chromosome (22). SOX9 has been shown to influence epithelial cell proliferation and migration in developing prostate and to have similar roles in prostate cancer. It is also a master factor regulating chondrocyte development (23, 24). SOX9 is upregulated in BCCs (25) and the Shh pathway downstream transcription factor, GLI-1, regulates SOX9 expression (26). SOX9 up-regulation in murine and human BCCs (FIGS. 3A-C) was confirmed. Genetic ablation of SOX9 reduced the expression of mTOR and the phosphorylation of p-p7056K and p-4E-BP1 (known downstream mTOR signaling proteins) and cyclin D1 (FIG. 3D, lanes 1 vs. 3) in ASZ001 cells to a level comparable to the inhibition of mTOR signaling by rapamycin (lanes 2 vs. 3).

### Example 5

### mTOR is a Direct Transcriptional Target of SOX9

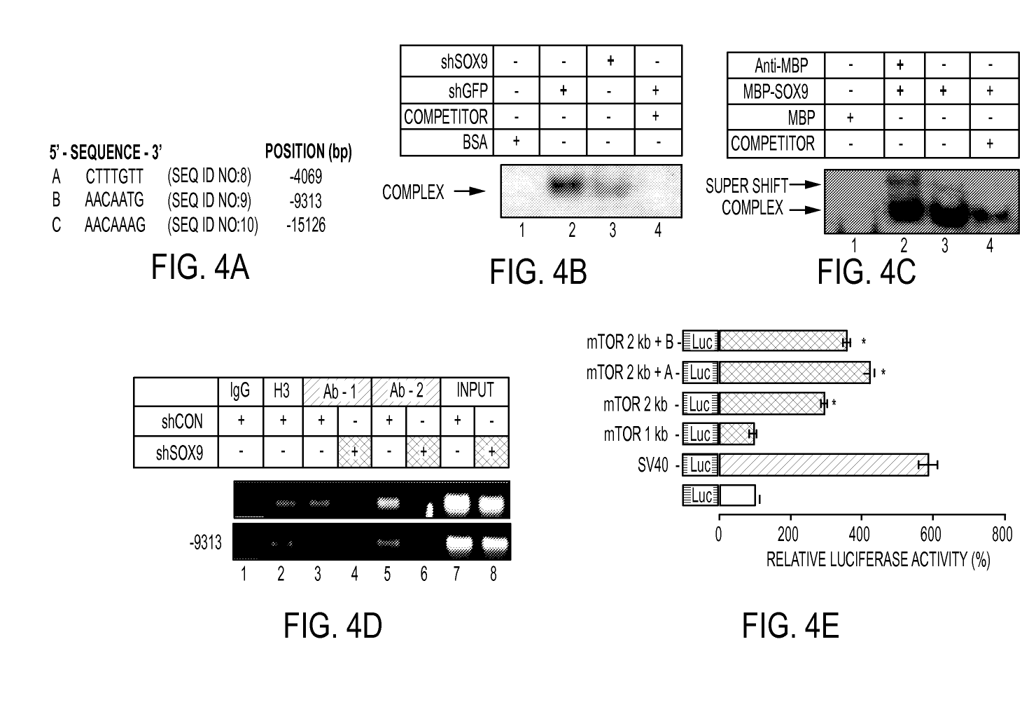

The apparent reduction in mTOR expression following pharmacological inhibition of the Shh pathway (FIG. 2) or SOX9 knockdown (FIGS. 3A-D) suggested that Shh signaling may transcriptionally regulate mTOR. It was found that the promoter region of mTOR possesses at least three SOX9 binding motifs (FIG. 4A). An electrophoretic mobility gel shift assay (EMSA) was performed using nuclear extracts prepared from ASZ001 nuclear extracts with labeled oligonucleotides containing the putative SOX9 binding motifs present in the mTOR promoter. A shifted band was detected which was totally displaced with a 100-fold excess of unlabeled oligonucleotides. shRNA-mediated knockdown of SOX9 in ASZ001 cells reduced the DNA binding capacity of ASZ001 nuclear extracts (FIG. 4B, lanes 2 vs. 3). Moreover, bacterially-produced and purified recombinant MBP-tagged SOX9 proteins bound directly to the SOX9 motif (mTOR-4069) (lane 3), which was super shifted in the presence of anti-MBP antibodies (lane 2) (FIG. 4C). SOX9 proteins occupy the mTOR promoter (−4069 and −9313) in ASZ001 cells, assessed by ChIP assay, and this interaction was lost following shRNA-mediated knockdown of SOX9 (FIG. 4D, lanes 3 vs. 4 and 5 vs. 6). To determine whether SOX9 directly regulates mTOR-dependent reporter activity, ASZ001 cells were transduced with mTOR promoter luciferase reporter constructs, which contain fragments of mTOR promoters, including 2 kb regions containing either 4069 or 9313 SOX9 binding sites, mTOR-A or mTOR-B, respectively. Basal mTOR-dependent reporter activity was detectable in the vector-transduced control cells and the presence of SOX9 binding motifs increased the reporter activity by 2.6-fold (−4069) and 3.2-fold (−9313) in cells transduced with MBP-SOX9, while no reporter activity was observed in cells transduced with truncated a 1 kb promoter fragment lacking SOX9 binding motifs (mTOR-1 kb) (FIG. 4E). These data indicate that the Shh pathway directly regulates mTOR via SOX9.

### Example 6

### Akt1 is Intrinsically Activated in Ptch1^{+/-} Postnatal Keratinocytes

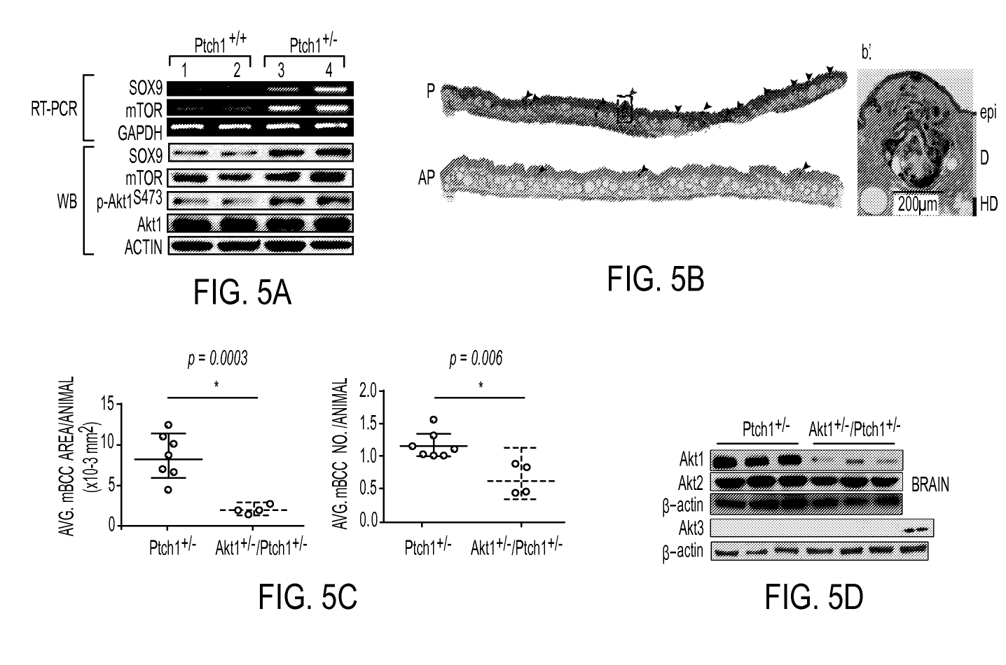

Phosphorylation of Akt at Ser473 by mTOR complex 2 (mTORC2) allows for its full activation (17, 27). To determine the impact of Shh pathway-dependent activation of mTOR signaling on Akt activation, the levels of Akt1 Ser473 phosphorylation in primary keratinocytes isolated from newborn Ptch1^{+/-}/SKH-1 mice were assessed thus avoiding extrinsic factors (e.g., UV) that could activate Akt. mTOR and Akt1 Ser473 phosphorylation were increased in Ptch1^{+/-} keratinocytes as compared to wild-type Ptch1^{+/+} keratinocytes (FIG. 5A) indicating that the Shh pathway intrinsically activates Akt.

### Example 7

### Akt1 Haplodeficiency Prevents the Formation of Spontaneous BCCs in Ptch1^{+/-}/SKH-1 Mice

Individuals with BCNS develop numerous spontaneous as well as UV-induced BCCs. The finding that Akt1 activation occurred in Ptch1^{+/-} cells, in the absence of external stimuli, posed the question whether intrinsic Akt1 activation is present in spontaneous BCCs seen in BCNS patients. To directly assess the role of Akt1 activation in vivo, Akt1 haplodeficient Ptch1^{+/-}/SKH-1 mice were generated. In Ptch1^{+/-}/SKH-1 mice, spontaneous microscopic BCCs are visible as early as 8 weeks of age with 100% tumor incidence (REF). FIG. 5B shows representative pictures of skin sections of Ptch1^{+/-}/SKH-1 (P) and Akt1^{+/-}/Ptch1^{+/-}/SKH-1 (AP) at 12 months of age. BCCs are shown by β-gal staining (black arrowheads) with an inset showing a magnified view of an area with multiple BCCs (red arrowhead, FIG. 5B). A significant reduction in both size and number of spontaneous BCCs were detected in Akt1^{+/-}/Ptch1^{+/-}/SKH-1 mice (n=4), compared to their Akt wild-type Ptch1^{+/-}/SKH-1 littermates (n=7) (FIG. 5C). The Akt isoforms share structural homology in mammals including humans, but display distinct physiological roles as well as tissue specificity (28). Among the three Akt isoforms, Akt1 and Akt2 (but not Akt3) are expressed in the skin. Although Akt1 has been the best-studied isoform relating to its role in carcinogenesis, in certain cases, Akt1 knockdown can upregulate and activate Akt2, which in turn compensates for Akt1 loss (29). The analysis of Akt1^{+/-}/Ptch1^{+/-}/SKH-1 mice shows that Akt2 levels were unchanged as compared to Ptch1^{+/-}/SKH-1 mice (FIG. 5D), indicating that Akt1 rather than Akt2 is involved the development of spontaneous BCCs.

### Example 8

### Akt1 Haplodeficiency Prevents UV-Induced BCC Development in Ptch1^{+/-}/SKH-1 Mice

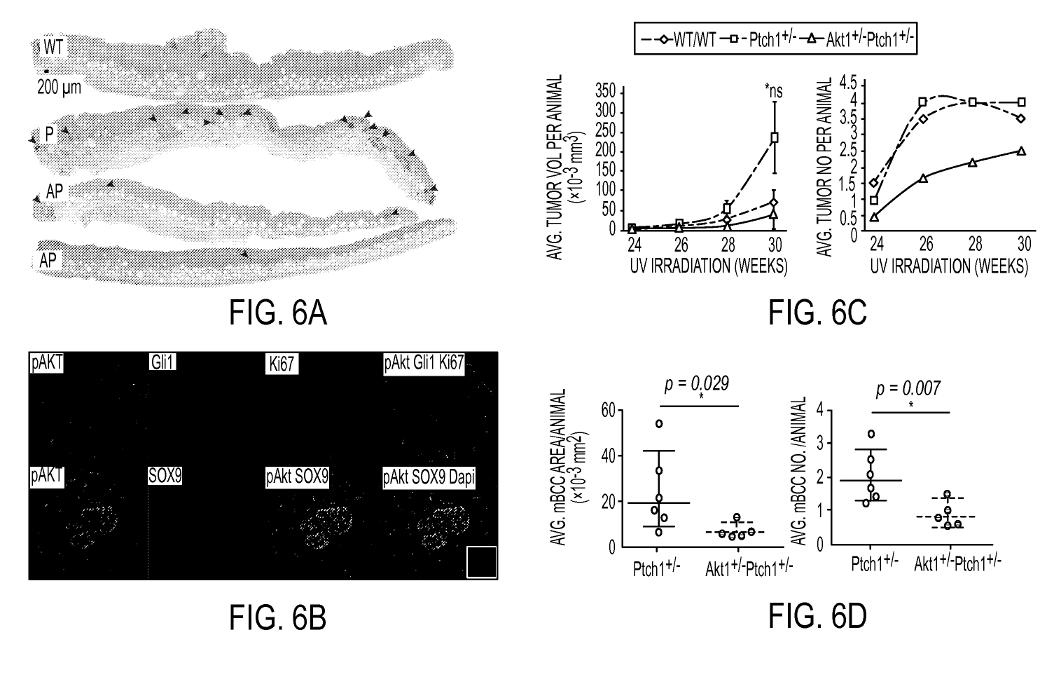

UV radiation is a complete carcinogen and UV exposure is the major known risk factor for the induction of BCCs, both in the general population and in BCNS patients. Sun-exposed areas develop more BCCs compared to sun-protected areas. While squamous cell carcinomas (SCCs) occur in chronically UV-irradiated skin of Ptch1^{+/+}/SKH-1 littermates (FIG. 6A, WT, red arrowheads; FIG. 6C), they are known to be less susceptible to the development of BCCs, as indicated by the absence of β-gal staining (FIG. 6A). In Ptch1^{+/-}/SKH-1 skin, chronic UV exposure resulted in extensive hyperplasia (P in FIG. 5B vs. P in FIG. 6A) and greatly enhanced the number and size of BCCs (ATHAR REF, FIG. 6A (P), black arrowheads). p-Akt1 S473 co-localizes with Gli1 or SOX9 in BCC cells (FIG. 6B) and genetic inhibition of Akt1 in BCC cells substantially reduced the development of UV-induced BCCs in Ptch1^{+/-}/SKH-1 mice (FIGS. 6A, AP; 6D), confirming the integral role of Akt1 activation in BCC pathogenesis.

### Example 9

### Pharmacological Inhibition of Akt Inhibits UV-Induced BCC Development

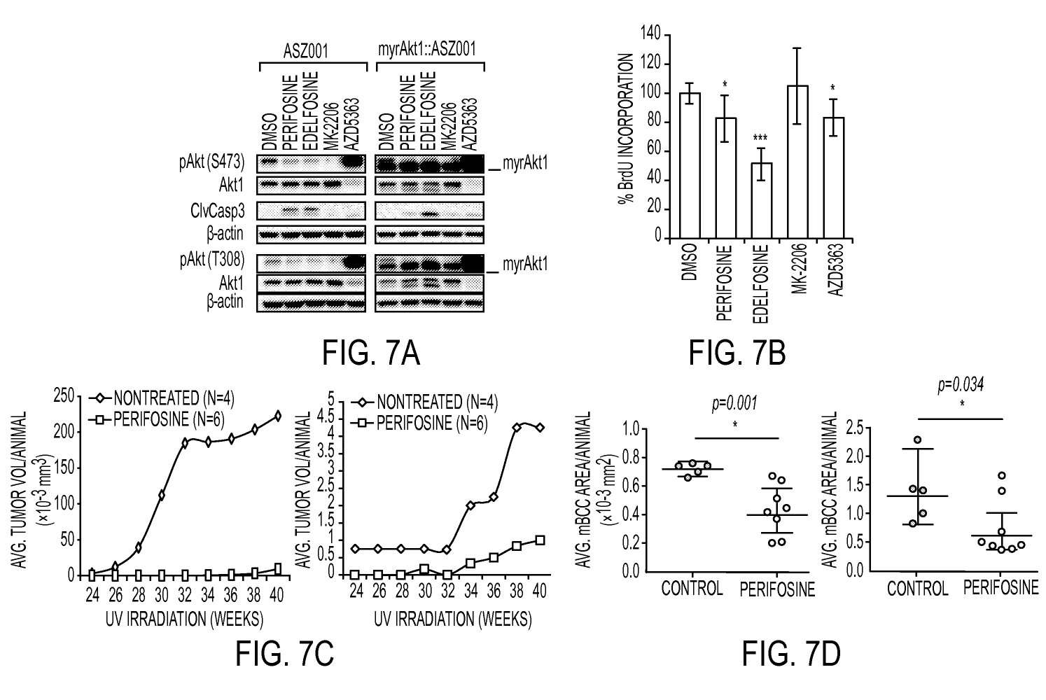

Akt signaling was inhibited pharmacologically in ASZ001 cells using Akt inhibitors that are currently in active clinical trials for various other human cancers. These include alkylphospholipid perifosine, MK-2206, an allosteric inhibitor which binds to and inhibits the Akt in a non-ATP competitive manner, and AZD5363, a pyrrolopyrimidine derivative that inhibits all Akt isoforms. Perifosine in particular was previously shown to be relatively nontoxic and well tolerated in Phase I/II clinical trials (30-34). The results of cytotoxic screening indicate that perifosine has a lower IC50 (4 μM), compared to other inhibitors which had IC50s ranging up to 50 μM (data not shown). Therefore, a concentration of 4 μM was selected for subsequent studies. Treatment of ASZ001 cells with perifosine and MK-2206 inhibited phosphorylation at S473 and T308 (FIG. 7A). AZD5363 treatment, however, increased phosphorylation at both sites (FIG. 7A), which is likely due to conformational changes that protect the sites from phosphatases (35-37). Nonetheless, Akt inhibition reduced proliferation of ASZ001 cells to varying degrees, assessed by BrdU incorporation after 24 hours of treatment with 4 μM of each Akt inhibitor (FIG. 7B). Perifosine and an alkyl-lysophospholipid analogue, edelfosine, were able to induce apoptosis in ASZ001 cells, as shown by an increase in cleaved caspase-3 (FIG. 7A, left panel). Overexpression of myrAkt1 protected ASZ001 cells from perifosine-induced apoptosis, as shown by decreased levels of caspase-3 (FIG. 7A, right panel). Oral administration of perifosine (125 mg/kg, twice weekly) to chronically UV-irradiated Ptch1^{+/-}/SKH-1 mice significantly reduced tumor burden (FIG. 7C) and size and number of microscopic BCCs (FIG. 7D). Perifosine is effective at preventing the growth of BCCs by enhancing tumor cell apoptosis.

### Example 10

### Perifosine Acts Synergistically in Combination with SMO Inhibitors in vitro and in vivo

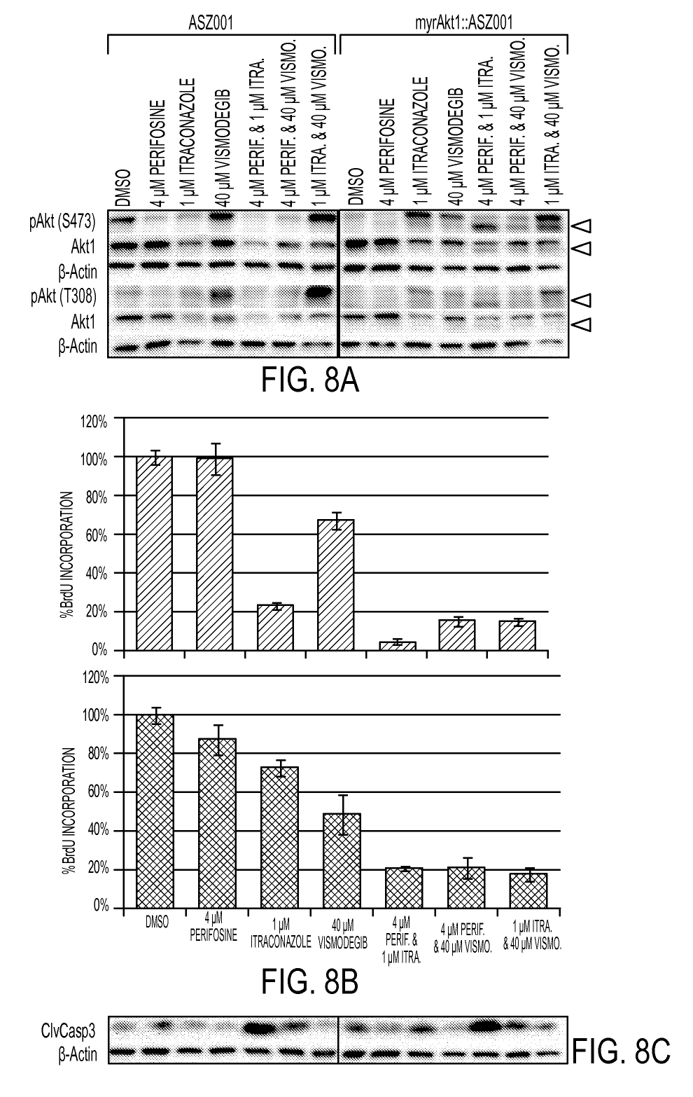

In vitro results: FIG. 8A shows that in both ASZ001 and myrAkt1-transformed ASZ001 cells, treatment regimens with Perifosine reduced phosphorylation of endogenous Akt at T308 and S473, (black arrowheads indicate myrAkt1Δ4-129). FIG. 8B further shows that in both ASZ001 (top) and myrAkt::ASZ001 (bottom), the combination regimen of perifosine with either Itraconazole or Vismodegib were more effective at inhibiting proliferation than single treatments alone. Itraconazole was less effective at inhibiting proliferation in transformed cells. Error bars represent SD. FIG. 8C) shows that increased apoptosis was detected in response to perifosine and was enhanced in combinations containing perifosine: however, myrAkt1::ASZ001s show less ClvCasp3, indicating an apparent protective effect.

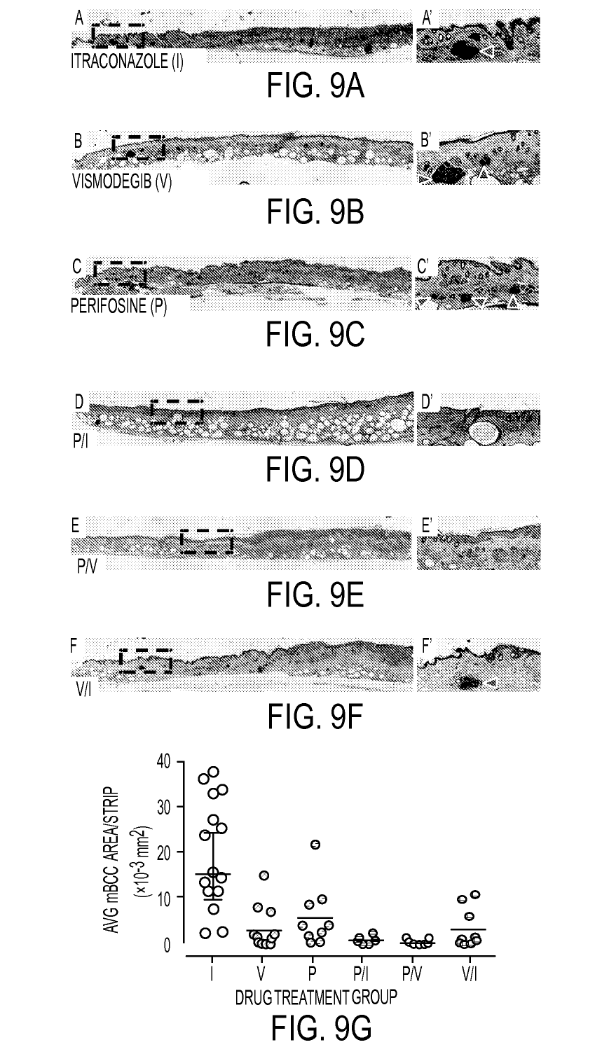

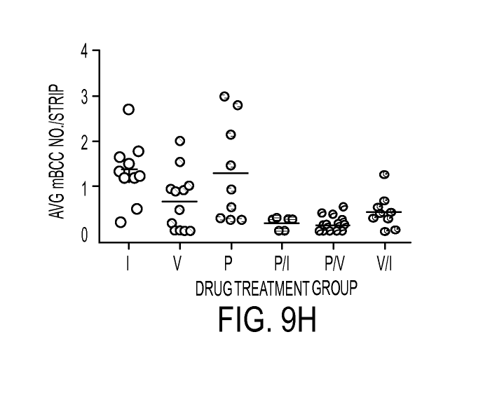

In vivo results: FIGS. 9 A-F shows slides of Ptch1^{+/-}/SKH-1 hairless mice that were UV-irradiated (180 mJ/cm2, twice weekly for 4 weeks) and treated with perifosine (50 mg/kg, PO BID) combined with itraconazole or vismodegib (100 mg/kg, PO BID). At the end of 4 weeks, dorsal skin samples were obtained and stained with β-galactosidase to identify BCCs (arrowheads). The slides show a dramatic reduction in the size of microscopic BCC. FIG. 9G shows that combination treatments with perifosine suppressed the size and frequency of microscopic BCCs (mBCC). Each dot represents data from one skin strip. Three skin strips (avg, 1.5 cm×0.1 cm) were analyzed for each mouse. Control treatment group average mBCC area/strip (90×10⁻³ mm2) and average mBCC number/strip (0.9) not shown.

TABLE 1
Hedgehog/Smoothened Inhibitors | Agonists | Antagonists (10)
Water-soluble

| Cat. No. | Product Name | Information |
|---|---|---|
| S1082 | Vismodegib (GDC- | Vismodegib (GDC-0449) is a potent, novel and |
| | 0449) | specific hedgehog inhibitor with IC50 of 3 nM and also |
| | | inhibits P-gp with IC50 of 3.0 μM in a cell-free assay. |
| S2151 | LDE225 (NVP- | LDE225 (NVP-LDE225, Erismodegib) is a Smoothened |
| | LDE225, Erismodegib) | (Smo) antagonist, inhibiting hedgehog (Hh) signaling with |
| | | IC50 of 1.3 nM (mouse) and 2.5 nM (human) in cell-free |
| | | assays, respectively. Phase 3. |
| S2157 | Taladegib | Taladegib (LY2940680) binds to the Smoothened (Smo) |
| | (LY2940680) | receptor and potently inhibits hedgehog (Hh) signaling. |
| | | Phase 1/2. |
| S3042 | Purmorphamine | Purmorphamine, which directly binds and activates |
| | | Smoothened, blocks BODIPY-cyclopamine binding to |
| | | Smo with IC50 of ~1.5 μM in HEK293T cell and also is |
| | | an inducer of osteoblast differentiation with EC50 of 1 μM. |
| S7160 | Glasdegib (PF- | Glasdegib (PF-04449913) is a potent, and orally |
| | 04449913) | bioavailable Smoothened (Smo) inhibitor with IC50 of 5 nM. |
| | | Phase 2. |
| S2777 | PF-5274857 | PF-5274857 is a potent and selective Smoothened (Smo) |
| | | antagonist, inhibits hedgehog (Hh) signaling with IC50 and |
| | | Ki of 5.8 nM and 4.6 nM, respectively, and can penetrate |
| | | the blood-brain barrier. |
| S8075 | GANT61 | GANT61 is an inhibitor for GLI1 as well as GLI2-induced |
| | | transcription inhibits hedgehog with IC50 of 5 μM in GLI1 |
| | | expressing HEK293T cell, displays selectivity over other |
| | | pathways, such as TNF and glucocorticoid receptor gene |
| | | transactivation. |
| S7092 | SANT-1 | SANT-1 directly binds to Smoothened (Smo) receptor with |
| | | Kd of 1.2 nM and inhibits Smo agonist effects with IC50 |
| | | of 20 nM. |
| S7138 | BMS-833923 | BMS-833923 is an orally bioavailable Smoothened |
| | | antagonist. Phase 2. |

TABLE 2
Shh pathway inhibitors
Cat. No. Product Name Information from APEXBIO

| | |
|---|---|
| 1. | A8659 SANT-1 Shh inhibitor, potent and cell-permeable |
| 2. | A8658 AY 9944 dihydrochloride hedgehog inhibitor |
| 3. | A8657 AZ 12080282 dihydrochloride hedgehog inhibitor |
| 4. | A8656 Jervine hedgehog signaling inhibitor |
| 5. | A8655 Ciliobrevin A Hh pathway antagonist |
| 6. | A8654 HPI 1 Hh signaling inhibitor |
| 7. | A8653 SANT-2 inhibitor of sonic hedgehog (Shh) signaling; |
| | antagonizes smoothened receptor activity (KD = 12 nM) |
| 8. | A8652 JK 184 Hh signaling inhibitor |
| 9. | A8651 LY2940680 hedgehog/SMO antagonist, inhibits Hh signaling |
| 10. | A8228 Purmorphamine hedgehog agonist |
| 11. | A8340 Cyclopamine hedgehog (Hh) signaling inhibitor |
| 12. | A8500 PF-5274857 SMO antagonist, potent and selective |

### REFERENCES

All references cited herein are hereby incorporated by reference in their entirety.
1. Briscoe J, and Therond P P. The mechanisms of Hedgehog signalling and its roles in development and disease. Nat Rev Mol Cell Biol. 2013; 14(7):416-29.
2. Kar S, Deb M, Sengupta D, Shilpi A, Bhutia S K, and Patra S K. Intricacies of hedgehog signaling pathways: A perspective in tumorigenesis. Experimental cell research. 2012; 318(16):1959-72.
3. Robbins D J, Fei D L, and Riobo N A. The Hedgehog Signal Transduction Network. Sci Signal. 2012; 5(246):re6-.
4. Jenkins D. Hedgehog signalling: emerging evidence for non-canonical pathways. Cell Signal. 2009; 21(7):1023-34.
5. Yun J I, Kim H R, Park H, Kim S K, and Lee J. Small molecule inhibitors of the hedgehog signaling pathway for the treatment of cancer. Archives of pharmacal research. 2012; 35(8):1317-33.
6. Miller D L, and Weinstock M A. Nonmelanoma skin cancer in the United States: incidence. J Am Acad Dermatol. 1994; 30(5 Pt 1):774-8.
7. Gorlin R J. Nevoid basal-cell carcinoma syndrome. Medicine. 1987; 66(2):98-113.
8. Tang J Y, Mackay-Wiggan J M, Aszterbaum M, Yauch R L, Lindgren J, Chang K, Coppola C, Chanana A M, Marji J, Bickers D R, et al. Inhibiting the hedgehog pathway in patients with the basal-cell nevus syndrome. The New England journal of medicine. 2012; 366(23):2180-8.
9. Kasper M, Jaks V, Hohl D, and Toftgard R. Basal cell carcinoma—molecular biology and potential new therapies. The Journal of clinical investigation. 2012; 122(2):455-63.
10. Wang G Y, Wang J, Mancianti M L, and Epstein E H, Jr. Basal cell carcinomas arise from hair follicle stem cells in Ptchc1(+/−) mice. Cancer Cell. 2011; 19(1):114-24.
11. Hanahan D, and Weinberg R A. The hallmarks of cancer. Cell. 2000; 100(1):57-70.
12. Eberl M, Klingler S, Mangelberger D, Loipetzberger A, Damhofer H, Zoidl K, Schnidar H, Hache H, Bauer H C, Solca F, et al. Hedgehog-EGFR cooperation response genes determine the oncogenic phenotype of basal cell carcinoma and tumour-initiating pancreatic cancer cells. EMBO molecular medicine. 2012; 4(3):218-33.
13. Yang W-L, Wu C-Y, Wu J, and Lin H-K. Regulation of Akt signaling activation by ubiquitination. Cell Cycle. 2010; 9(3):486-97.
14. Crowell J A, Steele V E, and Fay J R. Targeting the AKT protein kinase for cancer chemoprevention. Molecular cancer therapeutics. 2007; 6(8):2139-48.
15. Ming M, and He Y-Y. PTEN: New Insights into Its Regulation and Function in Skin Cancer. The Journal of investigative dermatology. 2009; 129(9):2109-12.
16. Ming M, Han W, Maddox J, Soltani K, Shea C R, Freeman D M, and He Y Y. UVB-induced ERK/AKT-dependent PTEN suppression promotes survival of epidermal keratinocytes. Oncogene. 2009; 29(4):492-502.
17. Sarbassov D D, Guertin D A, Ali S M, and Sabatini D M. Phosphorylation and regulation of Akt/PKB by the rictor-mTOR complex. Science (New York, N.Y. 2005; 307(5712):1098-101.
18. Kim K H, Back J H, Zhu Y, Arbesman J, Athar M, Kopelovich L, Kim A L, and Bickers D R. Resveratrol targets transforming growth factor-beta2 signaling to block UV-induced tumor progression. The Journal of investigative dermatology. 131(1):195-202.
19. Roessler E, Ermilov N, Grange D K, Wang A, Grachtchouk M, Dlugosz A A, and Muenke M. A previously unidentified amino-terminal domain regulates transcriptional activity of wild-type and disease-associated human GLI2. Human molecular genetics. 2005; 14(15):2181-8.
20. Kim J, Tang J Y, Gong R, Kim J, Lee J J, Clemons K V, Chong C R, Chang K S, Fereshteh M, Gardner D, et al. Itraconazole, a commonly used antifungal that inhibits Hedgehog pathway activity and cancer growth. Cancer Cell. 2010; 17(4):388-99.
21. Xie J, Aszterbaum M, Zhang X, Bonifas J M, Zachary C, Epstein E, and McCormick F. A role of PDGFRalpha in basal cell carcinoma proliferation. Proceedings of the National Academy of Sciences of the United States of America. 2001; 98(16):9255-9.
22. Lefebvre V, Dumitriu B, Penzo-Méndez A, Han Y, and Pallavi B. Control of cell fate and differentiation by Sry-related high-mobility-group box (Sox) transcription factors. The International Journal of Biochemistry & Cell Biology. 2007; 39(12):2195-214.
23. Wang H, Leav I, Ibaragi S, Wegner M, Hu G-f, Lu M L, Balk S P, and Yuan X. SOX9 Is Expressed in Human Fetal Prostate Epithelium and Enhances Prostate Cancer Invasion. Cancer research. 2008; 68(6):1625-30.
24. Murakami S, Kan M, McKeehan W L, and de Crombrugghe B. Up-regulation of the chondrogenic Sox9 gene by fibroblast growth factors is mediated by the mitogen-activated protein kinase pathway. Proceedings of the National Academy of Sciences. 2000; 97(3):1113-8.
25. Vidal V P, Ortonne N, and Schedl A. SOX9 expression is a general marker of basal cell carcinoma and adnexal-related neoplasms. Journal of Cutaneous Pathology. 2008; 35(4):373-9.
26. Bien-Willner G A, Stankiewicz P, and Lupski J R. SOX9cre1, a cis-acting regulatory element located 1.1 Mb upstream of SOX9, mediates its enhancement through the SHH pathway. Hum Mol Genet. 2007; 16(10):1143-56.
27. Shor B, Gibbons J J, Abraham R T, and Yu K. Targeting mTOR globally in cancer: thinking beyond rapamycin. Cell Cycle. 2009; 8(23):3831-7.
28. Zinda M J, Johnson M A, Paul J D, Horn C, Konicek B W, Lu ZH , Sandusky G, Thomas J E, Neubauer B L, Lai M T, et al. AKT-1, -2, and -3 are expressed in both normal and tumor tissues of the lung, breast, prostate, and colon. Clin Cancer Res. 2001; 7(8):2475-9.
29. Zhang L, Sun S, Zhou J, Liu J, Lv J H, Yu X Q, Li C, Gong L, Yan Q, Deng M, et al. Knockdown of akt1 promotes akt2 upregulation and resistance to oxidative-stress-induced apoptosis through control of multiple signaling pathways. Antioxidants & redox signaling. 2011; 15(1):1-17.
30. LoPiccolo J, Blumenthal G M, Bernstein W B, and Dennis P A. Targeting the PI3K/Akt/mTOR pathway: effective combinations and clinical considerations. Drug Resist Updat. 2008; 11(1-2):32-50.
31. Fu L, Kim Y A, Wang X, Wu X, Yue P, Lonial S, Khuri F R, and Sun S Y. Perifosine inhibits mammalian target of rapamycin signaling through facilitating degradation of major components in the mTOR axis and induces autophagy. Cancer Res. 2009; 69(23):8967-76.
32. Ghobrial I M, Roccaro A, Hong F, Weller E, Rubin N, Leduc R, Rourke M, Chuma S, Sacco A, Jia X, et al. Clinical and translational studies of a phase II trial of the novel oral Akt inhibitor perifosine in relapsed or relapsed/refractory Waldenstrom's macro globulinemia. Clin Cancer Res. 16(3): 1033 -41.
33. Pal S K, Reckamp K, Yu H, and Figlin R A. Akt inhibitors in clinical development for the treatment of cancer. Expert Opin Investig Drugs.
34. Gills J J, and Dennis P A. Perifosine: update on a novel Akt inhibitor. Curr Oncol Rep. 2009; 11(2):102-10.
35. Davies B R, Greenwood H, Dudley P, Crafter C, Yu D H, Zhang J, Li J, Gao B, Ji Q, Maynard J, et al. Preclinical pharmacology of AZD5363, an inhibitor of AKT: pharmacodynamics, antitumor activity, and correlation of monotherapy activity with genetic background. Molecular cancer therapeutics. 2012; 11 (4): 873-87.
36. Fox E M, Kuba M G, Miller T W, Davies B R, and Arteaga C L. Autocrine IGF-I/insulin receptor axis compensates for inhibition of AKT in ER-positive breast cancer cells with resistance to estrogen deprivation. Breast Cancer Res. 2013; 15(4):R55.
37. Liu P, Begley M, Michowski W, Inuzuka H, Ginzberg M, Gao D, Tsou P, Gan W, Papa A, Kim B M, et al. Cell-cycle-regulated activation of Akt kinase by phosphorylation at its carboxyl terminus. Nature. 2014; 508(7497):541-5.
38. Saldanha G. The Hedgehog signalling pathway and cancer. J Pathol. 2001; 193(4):427-32.
39. Athar M, Tang X, Lee J L, Kopelovich L, and Kim A L. Hedgehog signalling in skin development and cancer. Experimental Dermatology. 2006; 15(9):667-77.
40. Jiang J, and Hui C-c. Hedgehog Signaling in Development and Cancer. Developmental Cell. 2008; 15(6):801-12.
41. Shevde L A, and Samant R S. Nonclassical hedgehog-GLI signaling and its clinical implications. International journal of cancer. 2014; 135(1):1-6.
42. Javelaud D, Pierrat M-J, and Mauviel A. Crosstalk between TGF-fβ and hedgehog signaling in cancer. FEBS letters. 2012; 586(14):2016-25.
43. Schnidar H, Eberl M, Klingler S, Mangelberger D, Kasper M, Hauser-Kronberger C, Regl G, Kroismayr R, Moriggl R, Sibilia M, et al. Epidermal growth factor receptor signaling synergizes with Hedgehog/GLI in oncogenic transformation via activation of the MEK/ERK/JUN pathway. Cancer research. 2009; 69(4):1284-92.
44. Atwood S X, Li M, Lee A, Tang J Y, and Oro A E. GLI activation by atypical protein kinase C [igr]/[lgr] regulates the growth of basal cell carcinomas. Nature. 2013; 494(7438):484-8.
45. Das S, Samant R S, and Shevde L A. Nonclassical Activation of Hedgehog Signaling Enhances Multidrug Resistance and Makes Cancer Cells Refractory to Smoothened-targeting Hedgehog Inhibition. Journal of Biological Chemistry. 2013; 288(17):11824-33.
46. Xia Y P, Dai R L, Li Y N, Mao L, Xue Y M, He Q W, Huang M, Huang Y, Mei Y W, and Hu B. The protective effect of sonic hedgehog is mediated by the propidium iodide 3-kinase/AKT/Bcl-2 pathway in cultured rat astrocytes under oxidative stress. Neuroscience. 2012; 209(0):1-11.
47. Riobó N A, Lu K, Ai X, Haines G M, and Emerson C P. Phosphoinositide 3-kinase and Akt are essential for Sonic Hedgehog signaling. Proceedings of the National Academy of Sciences of the United States of America. 2006; 103(12):4505-10.
48. Shi G, Sohn K-C, Li Z, Choi D-K, Park Y M, Kim J-H, Fan Y-M, Nam Y H, Kim S, Im M, et al. Expression and Functional Role of Sox9 in Human Epidermal Keratinocytes. PLoS ONE. 2013; 8(1):e54355.
49. Vidal V P I, Ortonne N, and Schedl A. SOX9 expression is a general marker of basal cell carcinoma and adnexal-related neoplasms. Journal of Cutaneous Pathology. 2008; 35(4):373-9.
50. Kim A L, Athar M, Bickers D R, and Gautier J. Ultraviolet-B-induced G1 arrest is mediated by downregulation of cyclin-dependent kinase 4 in transformed keratinocytes lacking functional p53. The Journal of investigative dermatology. 2002; 118(5):818-24.
51. LeBel C P, Ischiropoulos H, and Bondy S C. Evaluation of the probe 2′,7′-dichlorofluorescin as an indicator of reactive oxygen species formation and oxidative stress. Chemical research in toxicology. 1992; 5(2):227-31.
52. Tang X, Kim A L, Feith D J, Pegg A E, Russo J, Zhang H, Aszterbaum M, Kopelovich L, Epstein E H, Jr., Bickers D R, et al. Ornithine decarboxylase is a target for chemoprevention of basal and squamous cell carcinomas in Ptch1+/−mice. The Journal of clinical investigation. 2004; 113(6): 867-75.

1. A method for treating basal cell carcinoma, basal cell nevus syndrome cancer, or XP BCC, comprising administering a therapeutically effective amount of an Akt inhibitor.

2. The method of claim 1, further comprising administering a therapeutically effective amount of an mTOR inhibitor or a sonic hedgehog pathway inhibitor.

3. The method of claim 1, further comprising administering a therapeutically effective amount of a sonic hedgehog pathway inhibitor and an mTOR inhibitor.

4. A method for treating a cancer that has activated sonic hedgehog and Akt, comprising administering a therapeutically effective amount of a sonic hedgehog pathway inhibitor and an Akt inhibitor, or a therapeutically effective amount of a sonic hedgehog pathway inhibitor and an mTOR inhibitor, a combination thereof.

5. A method for treating a cancer that has activated sonic hedgehog and mTOR cancer, comprising administering a therapeutically effective amount of a sonic hedgehog pathway inhibitor and an mTOR inhibitor, or sonic hedgehog inhibitor, an Akt inhibitor and an mTOR inhibitor.

6. The method of claim 1, wherein the Akt inhibitor is selected from the group consisting of perifosine, edelfosine, MK-2206 and AZD5363 or pharmaceutically active derivatives thereof.

7. The method of claim 2, wherein the sonic hedgehog pathway inhibitor is a smoothened inhibitor.

8. The method of claim 7, wherein the smoothened inhibitor is a member selected from the group consisting of vismodegib and itraconazole, an agent listed in Table 1 or Table 2, or pharmaceutically active derivatives thereof.

9. The method of claim 2, wherein the mTOR inhibitor is selected from the group consisting of rapamycin Agent OSI-027, XL765, everolimius, temsirolimus and zotarolimus, or pharmaceutically active derivatives thereof.

10. The method of claim 4, wherein the sonic hedgehog- and Akt-activated cancer is a member from the group consisting of colon, pancreas, medulloblastoma, prostate, esophageal, glioma, and gastrointestinal cancers.

11. The method of claim 5, wherein the sonic hedgehog- and mTOR- activated cancer is a member from the group consisting of colon, pancreas, medulloblastoma, prostate, esophageal, glioma, and gastrointestinal cancers.

12. A pharmaceutical composition, comprising a therapeutically effective amount of an Akt1 inhibitor together with a sonic hedgehog pathway inhibitor or a therapeutically effective amount of an Akt1 inhibitor, sonic hedgehog pathway inhibitor and an mTOR inhibitor.

13. (canceled)

14. The pharmaceutical composition of claim 12, wherein the Akt inhibitor is selected from the group consisting of perifosine, edelfosine, MK-2206 and AZD5363 or pharmaceutically active derivatives thereof.

15. The pharmaceutical composition of claim 12, wherein the sonic hedgehog pathway inhibitor is a smoothened inhibitor.

16. The pharmaceutical composition of claim 15, wherein the smoothened inhibitor is a member selected from the group consisting of vismodegib and itraconazole, an agent listed in Table 1 or Table 2, or pharmaceutically active derivatives.

17. The pharmaceutical composition of claim 12, formulated for topical or oral administration.

18. The pharmaceutical composition of claim 12, formulated for administration by injection.

19. The method of claim 1, wherein the dose is from about 0.1 mg/day to about 1 gm/day.

20. The method as of claim 1, wherein the of Akt inhibitor, is administered at a dose of about 1-25 mg/day, 25-50 mg/day, 50-100 mg/day, 100-200 mg/day, 200-300 mg/day, 400-500 mg/day and 500-1000 mg/day.

21. The method as of claim 2, wherein the sonic hedgehog pathway inhibitor is administered at a dose of about 1-25 mg/day, 25-50 mg/day, 50-100 mg/day, 100-200 mg/day, 200-300 mg/day, 400-500 mg/day and 500-1000 mg/day.

22. The method of claim 2, wherein the mTOR inhibitor is administered at a dose of about 1-25 mg/day, 25-50 mg/day, 50-100 mg/day, 100-200 mg/day, 200-300 mg/day, 400-500 mg/day and 500-1000 mg/day.

23. The pharmaceutical composition of claim 12, wherein the amount of sonic hedgehog inhibitor and the amount of Akt inhibitor respectively is from about 1-25 mg, 25-50 mg, 50-100 mg, 100-200 mg, 200-300 mg, 400-500 mg and 500-1000 mg.

24. The pharmaceutical composition of claim 13, wherein the amount of sonic hedgehog inhibitor, the amount of mTOR inhibitor and the amount of Akt inhibitor respectively is from about 1-25 mg, 25-50 mg, 50-100 mg, 100-200 mg, 200-300 mg, 400-500 mg and 500-1000 mg.


SEQUENCE LISTING (7 sequences)

SEQ ID NO: 1 - DNA, 36 bp, Artificial Sequence
  Synthetic oligonucleotide  pmTOR4069SOX9BE
tcaatggttttttgctttgtttctggagacagggcc

SEQ ID NO: 2 - DNA, 31 bp, Artificial Sequence
  Synthetic oligonucleotide  pmTOR9313 SOX9BE
aggctaattctgaacaatggtgagttctgtt

SEQ ID NO: 3 - DNA, 31 bp, Artificial Sequence
  Synthetic oligonucleotide  pmTOR15126 SOX9BE
ggaaagcaaagaaacaaagccactgacatac

SEQ ID NO: 4 - DNA, 20 bp, Artificial Sequence
  Synthetic oligonucleotide  mTOR9313IPF
gatggaaagggatgagcaga

SEQ ID NO: 5 - DNA, 20 bp, Artificial Sequence
  Synthetic oligonucleotide  mTOR9313IPR
ccaggatcgaactctccaac

SEQ ID NO: 6 - DNA, 20 bp, Artificial Sequence
  Synthetic oligonucleotide  mTOR4069IPF
gccaagagcttggaaaagaa

SEQ ID NO: 7 - DNA, 20 bp, Artificial Sequence
  Synthetic oligonucleotide  mTOR4069IPR
ctcttgggggtgctaggaat